(19) United States
(12) Patent Application Publication
(10) Pub. No.: US 2014/0303078 A1
(43) Pub. Date: Oct. 9, 2014

(54) MODULATION OF PANCREATIC BETA CELL PROLIFERATION

(71) Applicant: President and Fellows of Harvard College, Cambridge, MA (US)

(72) Inventors: Douglas A. Melton, Lexington, MA (US); Peng Yi, Arlington, MA (US)

(73) Assignee: President and Fellows of Harvard College, Cambridge, MA (US)

(21) Appl. No.: 14/125,276

(22) PCT Filed: Jun. 10, 2012
(86) PCT No.: PCT/US2012/041804

§ 371 Date: Jun. 20, 2014

Related U.S. Application Data
(60) Provisional application No. 61/495,868, filed on Jun. 10, 2011.
(60) Provisional application No. 61/613,856, filed on Mar. 21, 2012.

(51) Int. Cl. C07K 14/575; G01N 33/74; G01N 33/50

(52) U.S. Cl. CPC C07K 14/575; G01N 33/5023; G01N 33/74
USPC 514/6.7; 514/44 R; 514/9.7; 514/6.9; 435/6.13; 435/7.1; 435/6.12; 506/9; 536/23.51; 530/399; 536/23.1; 530/324

(57) ABSTRACT

Work described herein provides, in one embodiment, a method for increasing proliferation or replication of pancreatic beta cells in a subject in need thereof, comprising administering to said subject an effective amount of an agent that increases the level or activity of hepatocellular carcinoma-associated protein TD26 (TD26), thereby increasing proliferation or replication of pancreatic beta cells. Such an agent may function by, for example, increasing the level of active TD26 in the subject or by increasing the functional activity of TD26 in the subject.

### RELATED APPLICATIONS

This application claims the benefit of U.S. Provisional Application Ser. No. 61/495,868, filed Jun. 10, 2011, and U.S. Provisional Application Ser. No. 61/613,856, filed Mar. 21, 2012, the teachings of which are incorporated herein by reference in their entirety.

### GOVERNMENT SUPPORT

This invention was made with government support under DK090781 awarded by the National Institutes of Health. The government has certain rights in the invention.

### BACKGROUND OF THE INVENTION

Beta cells (β-cells) are a type of pancreatic cell located in the islets of Langerhans which make and secrete insulin, a hormone that controls the level of glucose in the blood. Beta cells can respond quickly to spikes in blood glucose by releasing stored insulin while simultaneously producing additional insulin for future needs. Impaired function and/or diminished numbers of beta cells are implicated in metabolic diseases including diabetes, obesity, and other disorders.

Diabetes is a disease derived from multiple causative factors and characterized by elevated levels of plasma glucose (hyperglycemia) in the fasting state. There are two main forms of diabetes mellitus: (1) insulin-dependent or Type 1 diabetes (a.k.a., Juvenile Diabetes) and (2) non-insulin-dependent or Type II diabetes (a.k.a., NIDDM).

Type 1 diabetes is caused by insulin deficiency resulting from loss of pancreatic beta cells, typically as a result of autoimmune destruction of the islets of Langerhans. Thus, in patients who suffer from type 1 diabetes the amount of insulin produced by the pancreatic islet cells is too low, resulting in elevated blood glucose levels (hyperglycemia). Patients with type 1 diabetes generally require lifelong insulin treatment, but even with frequent daily injections of insulin it is difficult to adequately control blood glucose levels. Treatments have been developed which can reduce immune system-mediated islet destruction; however, due to the relatively slow regeneration of human beta cells such treatments alone are not satisfactory means for improving the diabetic condition. These therapies could, however, be advantageously combined with therapeutic agent(s) capable of stimulating beta cell regeneration.

In type 2 diabetic patients, liver and muscle cells lose their normal ability to respond to normal blood insulin levels (insulin resistance), resulting in high blood glucose levels. Additionally, Type II diabetic patients exhibit impairment of beta cell function and an increase in beta cell apoptosis, causing a reduction in total beta cell mass over time. Eventually, the administration of exogenous insulin becomes necessary in type 2 diabetics.

Conventional methods for treating diabetes have included administration of fluids and insulin in the case of Type 1 diabetes and administration of various hypoglycemic agents in Type II diabetes. Unfortunately many of the known hypoglycemic agents exhibit undesirable side effects and toxicities. Thus, for both type 1 and type 2 diabetes, there is a need for development of agents capable of stimulating beta cell proliferation for use in therapeutic methods and formulations.

### SUMMARY OF THE INVENTION

Work described herein provides, in one embodiment, a method for increasing proliferation or replication of pancreatic beta cells in a subject in need thereof, comprising administering to said subject an effective amount of an agent that increases the level or activity of hepatocellular carcinoma-associated protein TD26 (TD26), thereby increasing proliferation or replication of pancreatic beta cells. Such an agent may function by, for example, increasing the level of active TD26 in the subject or by increasing the functional activity of TD26 in the subject.

Described herein, in one embodiment, is a method for treating or preventing a disorder associated with a reduced level of endogenous insulin in a subject comprising administering to said subject an effective amount of an agent that increases the level or activity of hepatocellular carcinoma-associated protein TD26 (TD26), thereby increasing proliferation of pancreatic beta cells and increasing the level of endogenous insulin in said subject. In one embodiment the agent is a TD26 protein or functional portion thereof or a nucleotide sequence encoding TD26 or a functional portion thereof.

Also described is a method for treating or preventing a disorder associated with resistance to endogenous insulin in a subject comprising administering to said subject an effective amount of an agent that increases the level or activity of hepatocellular carcinoma-associated protein TD26 (TD26), thereby increasing proliferation of pancreatic beta cells and increasing the level of endogenous insulin in said subject. In one embodiment the agent is a TD26 protein or functional portion thereof or a nucleotide sequence encoding TD26 or a functional portion thereof.

In particular embodiments, the administered agent increases the level of endogenous TD26 in said subject. In one aspect the agent increases expression of TD26. In another aspect the agent increases secretion of TD26. In yet another aspect the agent increases the stability of, or prevents or otherwise slows the degradation of TD26. It should be appreciated that the present invention contemplates any agent that is capable of increasing levels of TD26, or the half-life of TD26, in the subject.

In certain embodiments, the agent is TD26 protein or a functional portion thereof (e.g., a coiled-coil domain or a TD26 protein which lacks a native signal sequence). In certain embodiments the TD26 protein comprises SEQ ID NO: 1, SEQ ID NO: 2, SEQ ID NO: 3 or SEQ ID NO: 4. In some embodiments a functional portion can be, for example, a polypeptide which is less that the entire TD26 amino acid sequence, lacks the native signal sequence, and comprises the amino acid sequence of amino acids 22-76, 48-76, or 77-135 or SEQ ID NO: 1.

In other embodiments, the agent is a nucleic acid encoding TD26 protein or a functional portion of TD26. In certain embodiments the nucleic acid comprises all or a portion of SEQ ID NO: 14 or SEQ ID NO: 15.

In other embodiments the agent is an insulin receptor antagonist. For example, the agent can be selected from the group consisting of S661, a functional portion of S661, S961, a functional portion of S961, RB537, and a functional portion of RB537. In certain aspects the agent comprises all or a functional portion of SEQ ID NO: 16.

In some embodiments the insulin receptor antagonist is administered at a dose which preferably causes little or no increase in blood glucose levels, or causes only a transient increase in blood glucose levels. In the circumstance in which the dose causes an increase in blood glucose levels, it may be used in conjunction with an additional therapeutic agent to address the blood glucose level. As a non-limiting example, the insulin receptor antagonist may, in some instances, be administered to said subject at a dose of less than about 10 μMol/Kg/week (e.g., less than about 9 μMol/Kg/week, about 8 μMol/Kg/week, about 7 μMol/Kg/week, about 6 μMol/Kg/week, about 5 μMol/Kg/week, about 4 μMol/Kg/week, about 3 μMol/Kg/week, about 2 μMol/Kg/week, about 1 μMol/Kg/week, about 0.90 μMol/Kg/week, about 0.80 μMol/Kg/week, about 0.70 μMol/Kg/week, about 0.60 μMol/Kg/week, about 0.50 μMol/Kg/week, about 0.40 μMol/Kg/week, about 0.30 μMol/Kg/week, about 0.20 μMol/Kg/week, about 0.10 μMol/Kg/week). In a particular embodiment the insulin receptor antagonist is administered to said subject at a dose of about 1 μMol/Kg/week.

In some described embodiments the disorder is selected from the group consisting of diabetes (e.g., Type I diabetes or Type II diabetes), metabolic syndrome, glucose intolerance, and obesity.

Also disclosed is a method of identifying a candidate therapeutic agent for treatment of a disorder associated with a reduced level of endogenous insulin (e.g., an insulin receptor antagonist) comprising contacting a suitable cell with a test agent; and determining the effect of said test agent on level or activity of TD26, wherein a test agent which increases TD26 level or activity is a candidate therapeutic agent for treatment of a disorder associated with a reduced level of endogenous insulin. In some aspects the effect of said test agent on level or activity of TD26 is assessed by comparing the effect of said test agent against the effect of a therapeutic agent which is known to increase TD26 level or activity. One such therapeutic agent identified by work described herein is S961, for example. In some aspects the effect of said test agent on level or activity of TD26 is assessed by determining the effect of said test agent on gene expression level of TD26.

In other aspects, methods of increasing beta cell replication or proliferation by administering one or more insulin receptor antagonists are contemplated by the invention. Suitable antagonists may, for example, interfere with the ability of insulin to interact with the insulin receptor, or may neutralize the biological effects of insulin. Insulin receptor antagonists may, for example, increase the level or activity of TD26, preferably without markedly increasing blood glucose levels or only doing so transiently. In some embodiments, methods of treating diabetes by administering one or more insulin receptor antagonists are described herein.

It will be understood that all aspects of the invention are combinable with other aspects described herein, and that merely for brevity all possible combinations and permutations are not exhaustively listed. Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below for illustrative purposes. All publications, patent applications, patents, and other references mentioned herein are expressly incorporated by reference in their entirety. In cases of conflict, the present specification, including definitions, will control. The materials, methods and examples described herein are illustrative only and are not intended to be limiting. Other features and advantages of the invention will be apparent from and encompassed by the following detailed description and claims.

### BRIEF DESCRIPTION OF THE DRAWINGS

FIGS. 1A-1D show that the insulin receptor antagonist S961 induces beta cell replication. FIG. 1A shows treatment for 10 days with a control, and FIG. 1B shows treatment for 10 days with S961 (triple immunofluorescent staining using DAPI as marker for cell nuclei in blue, anti-insulin antibody staining as marker for beta cells in green, and anti-Ki67 (a marker of replication and cell proliferation) labeling the nuclei of proliferating cells in red). The white arrows point to replicating beta cells. FIG. 1C is a bar graph showing Ki67+/insulin+% after treatment for 7 days with increasing doses (from 0.125 μMol/Kg/week to 1 μMol/Kg/week) of S961. FIG. 1D is a bar graph showing significant increase in beta cell area/pancreas area after treatment for 7 days with increasing doses (from 0.125 μMol/Kg/week to 1 μMol/Kg/week) of S961. The control in these experiments is vehicle without S961. To count the Ki67+/insulin+ percentage and the beta cell area/pancreas area, the whole pancreas was cryosectioned and immunostained, and the standard graph analysis tools such as Metamorph and Photoshop were used for the quantification.

FIGS. 2A-2F show that the gene TD26, which is induced by the insulin receptor antagonist S961, is highly expressed in the liver and is a secreted protein. FIG. 2A shows gene expression microarray analysis of liver tissue after 7 days of treatment with S961. Genes close to the diagonal line show similar expression values in treated and untreated liver. Dots outside of the area between the thin red lines labeled “3 folds” represent genes significantly up- or down-regulated under treatment conditions. One of the upregulated genes, indicated by the black arrow, is EG624219 (the mouse ortholog of TD26). FIG. 2B shows relative expression of TD26 mRNA in mice treated with insulin receptor antagonist S961 for 7 days. TD26 expression increases in the liver and fat upon S961 treatment. FIG. 2C shows the predicted exon structure for the mouse ortholog of TD26. FIG. 2D shows an alignment of the sequences of TD26 protein with mouse and rat orthologs (human (Homo sapiens, SEQ ID NO: 1), mouse (Mus musculus, SEQ ID NO: 2) and rat (Rattus norvegicus, SEQ ID NO: 3)) along with a consensus sequence (SEQ ID NO: 4). The presumptive signal peptide is included at the N-terminus. FIG. 2E shows that Myc-tagged EG624219 (the mouse ortholog of TD26) and TD26 are expressed in Hepa1-6 cells (mouse hepatoma cells). Hepa1-6 cells have been transfected with a plasmid carrying the gene for TD26 fused to a Myc-tag. The cells were stained with myc antibody. FIG. 2F is a western blot illustrating, in 293T cells, that a myc-tagged mouse EG624219 (mouse TD26 protein) and a myc-tagged TD26 is secreted into the supernatant after 48 hrs, indicating that both the mouse ortholog of TD26 and human TD26 are secreted proteins.

FIGS. 3A and 3B show the expression of TD26 in human (FIG. 3A) and mouse (FIG. 3B) tissue based on tissue microarray data from the BioGPS online database (http://biogps.gnf.org) showing high and specific gene expression. FIG. 3A shows high expression in human liver, while FIG. 3B shows high expression in mouse brown adipose tissue, in liver and in pancreas.

FIGS. 4A-4C show that in vivo administration of EG624219 (mouse TD26 ortholog) DNA via injection into tail vein on day 1 increases beta cell replication. FIGS. 4A (control (green fluorescent protein (GFP))) and 4B (EG624219) show the results on day 9; Ki67 is a marker of replication. The green stain for insulin shows islets nested within the exocrine pancreas. The red dots in the islets show replicating beta cells. FIG. 4C is a bar graph showing Ki67+/insulin+% with control (GFP) as compared to EG624219. EG624219 injected animals demonstrated a 26 fold increase in beta cell replication compared to control injected animals (i.e., an average of 5.76% for EG624219 injected animals versus 0.22% for control injected animals).

FIG. 5 shows predicted sequence homologies for TD26 in various species including mouse (Mus musculus), dog (Canis familiaris), chicken (Gallus gallus), frog (Xenopus (Silurana) tropicalis), zebrafish (Danio rerio), opossum (Monodelphis domestica), monkey (Macaca mulatta), and human (Homo sapiens). From this analysis, TD26 appears to be a gene specific to mammals, as it is not found in chicken, frog, or fish.

FIG. 6 shows the sequence homology of human TD26 (“query,” including portion of predicted signal sequence) with human angiopoietin-related protein 3 precursor (“Sbjct”). TD26 (SEQ ID NO: 5) shows 22% identity and 49% homology to angiopoietin-related 3 precursor (SEQ ID NO: 6).

FIG. 7 shows the sequence homology of mouse TD26 ortholog (“query,” EG624219; SEQ ID NO: 7, which includes a portion of the predicted signal sequence) with Mus musculus angiopoietin-related protein 3 precursor (“Sbjct,” SEQ ID NO: 8).

FIG. 8 shows the sequence homology of angiopoietin-related protein 3 precursor (Homo sapiens; referred to as “angptl-3 Hs”) (SEQ ID NO: 9), angiopoietin-related protein 3 precursor (Mus musculus; referred to as “angptl-3 Mm”) (SEQ ID NO: 10), angiopoietin-related protein 4 precursor (Homo sapiens; referred to as “angptl-4 Hs”) (SEQ ID NO: 11), angiopoietin-related protein 4 precursor (Mus musculus; referred to as “angptl-4 Mm”) (SEQ ID NO: 12), TD26 ortholog (Mus musculus; EG624219) (SEQ ID NO: 2), and human TD26 (SEQ ID NO: 1).

FIG. 9 shows that both mouse and human TD26 proteins are predicted to have a signal sequence.

FIG. 10 shows that human TD26 is predicted to have a coiled-coiled structure.

FIG. 11 is a graph illustrating blood glucose levels (mg/dL) as a function of time after administration of various concentrations of S961, indicating that beta cell replication is increased upon administration of S961 without substantially impacting blood glucose levels.

FIG. 12 is a bar graph showing beta cell replication after repeated dosing of S661 in the pancreas of normal mice. Increased replication is shown as a fold increase compared to vehicle rate. Replication was analyzed by immunohistochemistry. The control in these experiments is a vehicle without S661.

FIG. 13 is a bar graph showing beta cell replication after repeated dosing of S661 in normal mice. Increased beta cell replication with S661 treatment is shown as a percentage of replication. Replication was analyzed by flow cytometry. The control in these experiments is a vehicle without S661. (*** p<0.001; Student's T-Test)

FIGS. 14A-14C show that in vivo administration of S661 increases beta cell replication in diet-induced obesity (DIO) mice. FIG. 14 (A) shows increased replication of beta cells and FIG. 14 (B) shows replication of non-beta cells after repeated dosing of S661 in pancreas in mice with diet-induced obesity. Increased beta cell replication with S661 treatment is shown as percent replication; there was no effect on non-beta cell replication. FIG. 14 (C) demonstrates that S661 treatment caused an increase in islet area relative to total pancreas area. Immunohistochemistry was performed to analyze replication and islet area. (* p<0.05; ** p<0.01; Student's T-Test)

FIG. 15 shows induction of beta cell replication by in vivo administration of plasmids encoding mouse TD26 polypeptide fragments via injection into tail vein. The control in these experiments was GFP-encoding plasmid.

### DETAILED DESCRIPTION OF THE INVENTION

Embodiments of the invention described herein arise from the observation that hepatocellular carcinoma-associated protein TD26 (herein referred to as “TD26”) induces pancreatic beta cell proliferation, as well as the observation that the insulin receptor antagonist S961 also induces pancreatic beta cell proliferation at low doses. The ability to modulate, and particularly to increase, beta cell function and cell mass, thereby increasing insulin secretion, provides modalities for treatment of, e.g., diabetes. Accordingly, work described herein provides methods of modulating pancreatic beta cell proliferation, serum insulin levels, levels of fatty acids, and blood glucose levels, as well as methods for treating and/or preventing disorders including diabetes, obesity and metabolic syndrome.

As used herein, the terms “beta cell,” “β-cell” or “pancreatic β-cell” include primary pancreatic β-cells, pancreatic β-like cells derived from dedifferentiated cells, e.g. from induced pluripotent stem cells (iPSCs), or pancreatic β-like cells that have been directly reprogrammed from a cell of another origin (e.g. a liver cell, fibroblast, or an exocrine pancreatic cell). In one embodiment, a β-cell is not an immortalized cell line (i.e., the β-cell does not proliferate indefinitely in culture). In one embodiment, the β-cell is not a transformed cell (i.e., the β-cell does not exhibit a transformation property, such as growth in soft agar, or absence of contact inhibition, to name just two examples).

As used herein, the term “endogenous pancreatic beta cell”, alternatively a “primary pancreatic beta cell” refers to an insulin producing cell of the pancreas of a mammal, or a cell of a pancreatic beta cell (beta cell) phenotype of a mammal. The phenotype of a pancreatic beta cell is well known by persons of ordinary skill in the art, and include, for example, secretion of insulin in response to an increase in glucose level, expression of markers such as c-peptide, PDX-1 polypeptide and Glut 2, as well as distinct morphological characteristics such as, but not necessarily, organized in islets in pancreas in vivo, and typically have small spindle like cells of about 9-15 μm diameter. Endogenous pancreatic beta cells can be found in the islets of Langerhans. In methods of the invention, the primary pancreatic beta cells can be contacted in vitro as part of the islets of Langerhans.

As used herein, the term “insulin producing cell” includes primary beta cells as that term is described herein, as well as pancreatic beta-like cells as that term is described herein, that synthesize (i.e., transcribe the insulin gene, translate the proinsulin mRNA, and modify the proinsulin mRNA into the insulin protein), express (i.e., manifest the phenotypic trait carried by the insulin gene), or secrete (release insulin into the extracellular space) insulin in a constitutive or inducible manner.

In one aspect the methods comprise contacting a cell with or administering to a subject a compound or agent that modulates TD26 protein level or activity. The term “modulates protein level or activity” refers to upregulation (activation or increasing activity) or downregulation (inhibition) of protein level, activity or function. In one embodiment, the modulation occurs by directly increasing or inhibiting the activity of a protein, i.e., via direct physical interaction with the protein. In one embodiment, the activity of the protein is modulated indirectly, for example, in signaling, by activating or inhibiting an upstream effector of the protein activity.

In particular aspects desirable compounds or agents increase levels or activity (e.g., by increasing expression and/or secretion) of TD26. Suitable compounds/agents include, but are not limited to, chemical compounds and mixtures of chemical compounds, e.g., small organic or inorganic molecules; saccharides; oligosaccharides; polysaccharides; biological macromolecules, e.g., peptides, proteins, and peptide analogs and derivatives; peptidomimetics; nucleic acids; nucleic acid analogs and derivatives; extracts made from biological materials such as bacteria, plants, fungi, or animal cells or tissues; naturally occurring or synthetic compositions; peptides; aptamers; and antibodies, or fragments thereof. A compound/agent can be a nucleic acid RNA or DNA, and can be either single or double stranded. Example nucleic acid compounds include, but are not limited to, a nucleic acid encoding a protein activator or inhibitor (e.g. transcriptional activators or inhibitors), oligonucleotides, nucleic acid analogues (e.g. peptide-nucleic acid (PNA), pseudo-complementary PNA (pc-PNA), locked nucleic acid (LNA) etc.), antisense molecules, ribozymes, small inhibitory or activating nucleic acid sequences (e.g., RNAi, shRNAi, siRNA, micro RNAi (mRNAi), antisense oligonucleotides etc.) A protein and/or peptide agent can be any protein that modulates gene expression or protein activity. Non-limiting examples include mutated proteins; therapeutic proteins and truncated proteins, e.g. wherein the protein is normally absent or expressed at lower levels in the target cell. Proteins can also be selected from genetically engineered proteins, peptides, synthetic peptides, recombinant proteins, chimeric proteins, antibodies (e.g., antibodies that interfere with interaction between insulin and the insulin receptor and increase the level or activity of TD26, with a resultant increase in beta cell replication), midibodies, minibodies, triabodies, humanized proteins, humanized antibodies, chimeric antibodies, modified proteins and fragments thereof. A compound or agent that increases expression of a gene or increases the level or activity of a protein encoded by a gene is also known as an activator or activating compound. A compound or agent that decreases expression of a gene or decreases the level or activity of a protein encoded by a gene is also known as an inhibitor or inhibiting compound.

In certain embodiments, agents that increase TD26 levels or activity include, but are not limited to, TD26 proteins or polypeptides (including both human TD26 and homologs thereof, and orthologous polypeptides and proteins from non-human species, e.g., mouse) and functional fragments thereof; nucleic acid molecules encoding TD26 proteins and polypeptides and functional fragments thereof; and insulin receptor antagonists.

Methods of the present invention contemplate the use of any insulin receptor antagonist. For example, insulin receptor antagonists may be agents that interfere with the ability of insulin to interact with the insulin receptor, as well as agents that are capable of neutralizing the biological effects of insulin, preferably without markedly increasing blood glucose levels or only doing so transiently). Those of skill in the art will understand that suitable insulin receptor antagonists are those which interfere with insulin signaling and are capable of increasing beta cell replication. In some embodiments, such insulin receptor antagonists increase the level or activity of TD26, preferably without markedly increasing blood glucose levels or doing so only transiently. It should be understood that suitable insulin receptor antagonists include, but are not limited to, any of the suitable compounds/agents described above that are capable of increasing the level or activity of TD26 and increasing beta cell replication, preferably without a marked non-transient increase in blood glucose levels, when administered to an individual.

In some aspects, an insulin receptor antagonist that increases beta cell replication can be a protein or peptide. In some embodiments, a protein or peptide insulin receptor antagonist increases the level or activity of TD26. It will be understood by those of skill in the art that the peptide insulin receptor antagonists of the present invention can be administered in their peptide form or as nucleic acids which encode the peptides and are capable of being translated in vivo to produce a peptide having the desired biological activity.

In some aspects, a peptide insulin receptor antagonist comprises a first functional portion, a second functional portion, and a linker therebetween, preferably engineered for flexibility and/or solubility.

The first and second functional portions can comprise, for example, amino acid sequences which exhibit an affinity for the insulin receptor that is greater than or equal to the affinity of insulin for the insulin receptor (e.g., affinity-optimized sites).

In some embodiments, the first functional portion comprises a peptide having the amino acid sequence of SEQ ID NO: 18. In some embodiments, the first functional portion comprises a peptide having an amino acid sequence that is at least 80% identical to SEQ ID NO: 18, and retains the desired biological activity.

In some embodiments, the linker comprises a flexible linker. In some embodiments, the linker comprises a soluble linker. In some embodiments, the linker comprises an amino acid linker. In some embodiments, the amino acid linker has one or more amino acid residues. In other embodiments, the amino acid linker has up to seven amino acid residues. In some embodiments, the linker comprises one or more glycine residues and one or more serine residues. In some embodiments, the linker comprises one or more GGS repeats (e.g., GGS, GGSGGS, GGSGGSGGS, etc.). In one embodiment, a flexible linker comprises SEQ ID NO: 19. In certain embodiments, the flexible linker comprises an ethylene glycol-based linker as described in Schäffer et al., (Schäffer et al., PNAS 100(8):4435-4439 (2003), incorporated herein by reference in its entirety). In an embodiment, the linker comprises a triethylene glycol-based linker.

In some embodiments, the second functional portion comprises a peptide having the amino acid sequence of SEQ ID NO: 20. In some embodiments, the second functional portion comprises a peptide having an amino acid sequence that is at least 80% identical to SEQ ID NO: 20, and retains the desired biological activity. In some embodiments, the second functional portion comprises a peptide having amino acid sequence SLEEEWAQIQCEVWGRGCPSY (SEQ ID NO: 23). In some embodiments, the second functional portion comprises a peptide having an amino acid sequence that is at least 80% identical to SEQ ID NO: 23, and retains the desired biological activity. In some embodiments, the second functional portion comprises a peptide having amino acid sequence L-Xaa-Xaa-EWA-Xaa-Xaa-QCEV-Xaa-GRGCPS (SEQ ID NO: 24), wherein Xaa is any amino acid. In some embodiments, the second functional portion comprises a peptide having an amino acid sequence that is at least 80% identical to SEQ ID NO: 24, and retains the desired biological activity.

In some embodiments, a peptide insulin receptor antagonist that increases beta cell replication comprises peptide S961 (SEQ ID NO: 16, with an acid C-terminus). In some embodiments, peptide S961 increases the level or activity of TD26, preferably without markedly increasing blood glucose levels, or doing so only transiently.

In some aspects the peptide S961 (or a functional portion thereof) or a nucleic acid encoding peptide S961 comprises a variant sequence. The variant sequence can include one or more sequence variations provided that such variations do not eliminate the effect of increasing beta cell replication. In some embodiments, the sequence variations comprise conservative variations. In preferred embodiments, a nucleic acid encoding a S961 peptide of the present invention comprises a nucleotide sequence at least 80% identical to a nucleic acid encoding S961.

In some embodiments, a S961 peptide of the present invention comprises an amino acid sequence at least 80% identical to a S961 peptide. In some aspects, said S961 peptide comprises an amino acid sequence at least 80% identical to SEQ ID NO: 16. In some aspects, said peptide comprises an amino acid sequence at least 80% identical to SEQ ID NO: 18. In some embodiments, said peptide comprises an amino acid sequence at least 80% identical to SEQ ID NO: 23. In some embodiments, said peptide comprises an amino acid sequence at least 80% identical to, in order from N-terminus to C-terminus, SEQ ID NO: 18, SEQ ID NO: 19 and SEQ ID NO: 23, with an acid C-terminus.

In some embodiments, a peptide insulin receptor antagonist that increases beta cell replication comprises peptide S661 (SEQ ID NO: 16, with an amide C-terminus). In some embodiments, peptide S661 increases the level or activity of TD26, preferably without markedly increasing blood glucose levels or doing so only transiently. In some aspects the peptide S661 (or a functional portion thereof) or a nucleic acid encoding peptide S661 comprises a variant sequence provided that such variations do not eliminate the effect of increasing beta cell replication. In some embodiments, the sequence variations comprise conservative variations. In preferred embodiments, a nucleic acid encoding a S661 peptide of the present invention comprises a nucleotide sequence at least 80% identical to a nucleic acid encoding S661.

In some embodiments, a S661 peptide of the present invention comprises an amino acid sequence at least 80% identical to a S661 peptide. In some aspects, said S661 peptide comprises an amino acid sequence at least 80% identical to SEQ ID NO: 16. In some aspects, said peptide comprises an amino acid sequence at least 80% identical to SEQ ID NO: 18. In some embodiments, said peptide comprises an amino acid sequence at least 80% identical to SEQ ID NO: 23. In some embodiments, said peptide comprises an amino acid sequence at least 80% identical to, in order from N-terminus to C-terminus, SEQ ID NO: 18, 19 and 23, with an amide C-terminus.

In some embodiments, a peptide insulin receptor antagonist that increases beta cell replication comprises peptide RB537 (SEQ ID NO: 17). In some embodiments, peptide RB537 increases the level or activity of TD26, preferably without markedly increasing blood glucose levels, or doing so only transiently. In some aspects the peptide RB537 (or a functional portion thereof) or a nucleic acid encoding peptide RB537 comprises a variant sequence provided that such variations do not eliminate the effect of increasing beta cell replication. In some embodiments, the sequence variations comprise conservative variations. In preferred embodiments, a nucleic acid encoding a RB537 peptide of the present invention comprises a nucleotide sequence at least 80% identical to a nucleic acid encoding RB537.

In some embodiments, a RB537 peptide of the present invention comprises an amino acid sequence at least 80% identical to a RB537 peptide. In some aspects, said RB537 peptide comprises an amino acid sequence at least 80% identical to SEQ ID NO: 17. In some embodiments, said RB537 peptide comprises an amino acid sequence at least 80% identical to SEQ ID NO: 18. In some embodiments, said RB537 peptide comprises an amino acid sequence at least 80% identical to SEQ ID NO: 20. In some embodiments, said RB537 peptide comprises an amino acid sequence at least 80% identical, from N-terminus to C-terminus, to SEQ ID NO: 18, SEQ ID NO: 19, and SEQ ID NO: 20.

In some embodiments, an insulin receptor antagonist that increases beta cell replication comprises an antibody. In some embodiments, an insulin receptor antagonist that increases beta cell replication comprises an antibody that binds to the insulin receptor. In some embodiments, an insulin receptor antagonist antibody increases the level or activity of TD26, preferably without markedly increasing blood glucose levels or doing so only transiently. As used herein, “antibody” is used in the broadest sense and includes fully assembled antibodies, tetrameric antibodies, monoclonal antibodies, polyclonal antibodies, multispecific antibodies (e.g., bispecific antibodies), antibody fragments that can bind an antigen (e.g., Fab′, F′(ab)2, Fv, single chain antibodies, diabodies), and recombinant peptides comprising any of the above as long as they exhibit the desired biological activity. An “immunoglobulin” or “tetrameric antibody” is a tetrameric glycoprotein that consists of two heavy chains and two light chains, each comprising a variable region and a constant region. Antigen-binding portions may be produced by recombinant DNA techniques or by enzymatic or chemical cleavage of intact antibodies. Antibody fragments or antigen-binding portions include Fab, Fab′, F(ab′).sub.2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), a antigen-binding-domain immunoglobulin fusion protein, a camelized antibody, a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity. “Antibody variant” refers to an antibody polypeptide sequence that contains at least one amino acid substitution, deletion, or insertion in the variable region of the natural antibody variable region domains. Variants may be substantially homologous or substantially identical to the unmodified antibody. A “chimeric antibody” refers to an antibody containing sequence derived from two different antibodies (see, e.g., U.S. Pat. No. 4,816,567) which typically originate from different species. Most typically, chimeric antibodies comprise human and rodent antibody fragments, generally human constant and mouse variable regions.

Examples of insulin receptor antagonists which may be suitable for use in the present invention, in some embodiments, may include anti-insulin receptor antibodies reported in the literature (see, e.g., Roth et al., J Biol Chem. 1983 Oct. 25; 258(20):12094-7; Morgan et al., Proc Natl Acad Sci USA. 1986 January; 83(2):328-32; Taylor et al., Biochem J. 1987 Feb. 15; 242(1):123-9; Nagy et al., Endocrinology. 1990 January; 126(1):45-52; and Fujita et al., Acta Diabetol. 2002 December; 39(4):221-7) which exhibit the desired biological activity. In some embodiments, anti-insulin receptor antibodies may increase the level or activity of TD26, preferably without markedly increasing blood glucose levels, or doing so only transiently.

In some embodiments, the present invention contemplates polynucleotides encoding antibodies and peptides of the invention. The present invention also contemplates vectors comprising such polynucleotides, host cells comprising such polynucleotides or vectors, and methods of producing antibodies and polypeptides of the invention comprising growing such host cells in culture medium under suitable conditions and optionally isolating the encoded antibody or polypeptide from the host cells or culture medium, optionally followed by further purification of the antibody or polypeptide. Antibody isolation and purification methods are well within the level of ordinary skill in the art.

In certain embodiments, an insulin receptor antagonist that is capable of increasing beta cell replication comprises a chemical compound, such as a low molecular weight organic molecule, for example. In some embodiments, the chemical compound increases the level or activity of TD26.

The terms “increased,” “increase,” “enhance” or “activate” are all used herein to generally mean an increase by a significant amount. In some embodiments of this and other aspects of the invention, level or activity of the TD26 protein is increased by at least 5%, at least 10%, at least 20%, at least 30%, at least 40%, at least 50%, at least 60%, at least 70%, at least 80%, at least 90%, at least 1-fold, at least 1.1-fold, at least 1.5-fold, at least 2-fold, at least 3-fold, at least 4-fold, at least 5-fold, or more relative to a control. In some embodiments of this and other aspects of the invention, the activator of protein activity has an EC50 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM. Protein activity can be measured by means well known to those of skill in the art and may be measured by different methods or assays depending on context.

In other aspects desirable compounds or agents decrease levels or activity (e.g., by decreasing expression and/or secretion) of TD26. Decreasing expression and/or secretion of TD26 may be desirable for treating disorders characterized by excessive insulin levels, which may be exacerbated by increased TD26 levels or activity, such as insulinomas, for example.

The terms “decrease,” “reduced,” “reduction,” “decrease” or “inhibit” are all used herein generally to mean a decrease by a significant amount. In some embodiments of this and other aspects of the invention, level or activity of the protein encoded by the gene is inhibited or lowered by at least 5%, at least 10%, at least 20%, at least 30%, at least 40%, at least 50%, at least 60%, at least 70%, at least 80%, at least 90%, at least 95%, at least 98%, or 100% (e.g. complete loss of activity) relative to a control. In some embodiments of this and other aspects of the invention, the inhibitor has an 1050 of less than or equal to 500 nM, less than or equal to 250 nM, less than or equal to 100 nM, less than or equal to 50 nM, less than or equal to 10 nM, less than or equal to 1 nM, less than or equal to 0.1 nM, less than or equal to 0.01 nM, or less than or equal to 0.001 nM. Compounds that decrease TD26 levels or activity include, for example, anti-TD26 antibodies, antisense molecules which target TD26, and siRNA molecules which target TD26.

TD26 proteins and polypeptides suitable for use in the present invention include, for example, human TD26 proteins and polypeptides. A human TD26 protein sequence has been deposited with GENBANK™ as Accession No. NP 061157.3. The amino acid sequence of human TD26 protein is:

MPVPALCLLWALAMVTRPASAAPMGGPELAQHEELTLLFHGTLQLGQAL
NGVYRTTEGRLTKARNSLGLYGRTIELLGQEVSRGRDAAQELRASLLET
QMEEDILQLQAEATAEVLGEVAQAQKVLRDSVQRLEVQLRSAWLGPAYR
EFEVLKAHADKQSHILWALTGHVQRQRREMVAQQHRLRQIQERLHTAAL
PA (SEQ ID NO: 1; predicted signal sequence
underlined).

Accordingly, in some embodiments the methods described herein include contacting a cell or culture medium with or administering to a subject the polypeptide of SEQ ID NO: 1 or a fragment, e.g., a functional portion, thereof (or a nucleic acid encoding the polypeptide or fragment). As used herein, a functional portion or fragment of TD26 is one which increases beta cell replication, for example, upon administration to a mammal or in a test animal. Suitable fragments include, for example, the polypeptide of SEQ ID NO: 1 lacking a native signal sequence and polypeptide portions of SEQ ID NO: 1 comprising a coiled-coil domain (CCD).

In certain embodiments, a functional portion of a TD26 polypeptide or protein useful for increasing the level or activity of TD26 and/or increasing beta cell replication in vivo lacks one or more domains (with reference to SEQ ID NO: 1, for example).

In some embodiments, a functional portion of TD26 lacks an LPL domain. In some embodiments, a functional portion of TD26 lacks one or more CCD (for example, lacks the first and/or second CCD). In certain aspects the polypeptide may lack the entire domain, while in other aspects the polypeptide may lack an intact (complete) domain (i.e., may contain a portion of the domain). In certain aspects the polypeptide may lack a functional domain (e.g., a functional LPL domain or a functional CCD).

Alternatively, a functional portion of a TD26 polypeptide or protein useful for increasing the level or activity of TD26 and/or beta cell replication in vivo comprises one or more domains (e.g., an intact domain or a functional domain) of the TD26 protein. In some embodiments, a functional portion of TD26 comprises the LPL domain. In some embodiments, a functional portion of TD26 comprises one or more CCD (e.g., the first CCD and/or the second CCD). In certain embodiments a functional portion of TD26 comprises some or all of the amino acid sequence between the first and second CCD of TD26 (“the intervening sequence” or “IVS”).

In certain embodiments, a functional portion of TD26 does not comprise the native signal sequence or the complete amino acid sequence or nucleotide sequence of TD26 or the functional portion does not include the complete amino acid sequence of TD26 lacking its signal peptide or a nucleic acid encoding the complete amino acid sequence of TD26 lacking its signal peptide. It will be understood that in many secreted proteins the signal sequence is cleaved and is not part of the polypeptide sequence of the final protein.

In an embodiment, a functional portion of a TD26 polypeptide or protein useful for increasing beta cell replication in vivo comprises a peptide of amino acid 22 to 76 of the polypeptide of SEQ ID NO: 1. In some embodiments, a functional portion of a TD26 polypeptide or protein comprises a peptide of amino acid 22 to 76 of the polypeptide of SEQ ID NO: 1 comprising one or more conservative amino acid substitutions, provided that the peptide retains the ability to increase beta cell replication.

In an embodiment, a functional portion of a TD26 polypeptide or protein useful for increasing beta cell replication in vivo comprises an LPL domain, but lacks a CCD1, IVS, and CCD2. In an embodiment, a functional portion of a TD26 polypeptide or protein useful for increasing beta cell replication in vivo comprises a predicted LPL domain, but lacks a predicted CCD1, a predicted IVS, and a predicted CCD2.

In an embodiment, a functional portion of a TD26 polypeptide or protein useful for increasing beta cell replication in vivo comprises a peptide of amino acid 48 to 76 of the polypeptide of SEQ ID NO: 1. In some embodiments, a functional portion of a TD26 polypeptide or protein comprises a peptide of amino acid 48 to 76 of the polypeptide of SEQ ID NO: 1 comprising one or more conservative amino acid substitutions, provided that the peptide retains the ability to increase beta cell replication.

In an embodiment, a functional portion of a TD26 polypeptide or protein useful for increasing beta cell replication in vivo lacks an intact LPL domain as well as a CCD1, a IVS, and a CCD2. In one embodiment, a functional portion of a TD26 polypeptide or protein useful for increasing beta cell replication in vivo comprises an amino acid of SEQ ID NO: 1 lacking an intact LPL domain, a CCD1, a IVS, and a CCD2. In an embodiment, a functional portion of a TD26 polypeptide or protein useful for increasing beta cell replication in vivo lacks an intact predicted LPL domain as well as a predicted CCD1, a predicted IVS, and a predicted CCD2.

In an embodiment, a functional portion of a TD26 polypeptide or protein useful for increasing beta cell replication in vivo comprises a peptide of amino acid 77 to 135 of the polypeptide of SEQ ID NO: 1. In some embodiments, a functional portion of a TD26 polypeptide or protein comprises a peptide of amino acid 77 to 135 of the polypeptide of SEQ ID NO: 1 comprising one or more conservative amino acid substitutions, provided that the peptide retains the ability to increase beta cell replication.

In an embodiment, a functional portion of a TD26 polypeptide or protein useful for increasing beta cell replication in vivo comprises a CCD1, but lacks a CCD2, a IVS, and a LPL domain. In an embodiment, a functional portion of a TD26 polypeptide or protein useful for increasing beta cell replication in vivo comprises a predicted CCD1, but lacks a predicted CCD2, a predicted IVS, and a predicted LPL domain.

Those skilled in the art will understand that the description of functional portions of TD26 described above with respect to SEQ ID NO: 1 are illustrative only. For example, such description applies similarly to SEQ ID NOs 2-4.

In certain embodiments, a functional polypeptide of SEQ ID NO: 1 comprises a protein sequence in which the signal sequence is replaced with a sequence that is capable of directing secretion of the polypeptide, such as a human growth hormone signal peptide, for example. In other embodiments, polypeptides and proteins suitable for use in the invention include the polypeptides of SEQ ID NO: 2, SEQ ID NO: 3, and SEQ ID NO: 4, as well as functional fragments thereof. Other suitable polypeptides and proteins can be identified by comparison with the amino acid sequence of human TD26 to identify polypeptides and proteins sharing significant sequence identity or homology with human TD26. Identity or homology may be present across the entire protein or polypeptide or may be present only or primarily across particular domains of the protein or polypeptide (e.g., across a CCD domain or other functional domain). Computerized algorithms for conducting such sequence comparisons are known in the art, and exemplary methods have been used in the work described herein. For example, computer algorithm analysis of amino acid sequence (and nucleic acid sequence) homology may include the utilization of any number of available software packages, such as, for example, the BLAST, DOMAIN, BEAUTY (BLAST Enhanced Alignment Utility), GENPEPT and TREMBL packages.

Useful nucleic acid molecules and their encoded polypeptides refer to all forms of nucleic acids of a respective gene (e.g., gene, pre-mRNA, mRNA) or proteins, their polymorphic variants, alleles, mutants, and interspecies homologs that (as applicable to nucleic acid or protein): (1) have an amino acid sequence that has greater than about 80% amino acid sequence identity, or greater than about 85%, about 90%, about 91%, about 92%, about 93%, about 94%, about 95%, about 96%, about 97%, about 98% or about 99% or greater amino acid sequence identity, preferably over a region of at least about 20, 25, 30, 35, 40, 45, 50, 75 or more amino acids, to a polypeptide described herein; (2) specifically bind to antibodies, e.g., polyclonal antibodies, raised against an immunogen comprising a reference amino acid sequence, immunogenic fragments thereof, and conservatively modified variants thereof; (3) specifically hybridize under stringent hybridization conditions to a nucleic acid encoding a reference amino acid sequence, and conservatively modified variants thereof; (4) have a nucleic acid sequence that has greater than about 80%, preferably greater than about 85%, 90%, 95%, 96%, 97%, 98%, 99%, or higher nucleotide sequence identity, preferably over a region of at least about 20, 25, 30, 35, 40, 45, 50, 75, 100, 200 or more nucleotides, to a reference nucleotide sequence described herein. In some embodiments the reference nucleotide sequence will lack the portion encoding the signal sequence.

In some aspects the TD26 nucleic acid (i.e., a nucleic acid encoding a TD26 polypeptide or functional portion thereof) or TD26 protein sequence comprises a variant sequence. The variant sequence can include one or more naturally occurring sequence variations. Non-limiting examples of naturally occurring sequence variations include one or more of the single nucleotide polymorphisms, or amino acid variations, identified in Table 1 below. Thus, TD26 sequences of the invention can comprise naturally occurring (as well as non-naturally occurring) variations provided that such variations do not eliminate the beta cell proliferative effect of the TD 26 sequence. In some embodiments, the sequence variations comprise conservative variations. In preferred embodiments, a nucleic acid encoding a TD26 protein of the present invention comprises a nucleotide sequence at least 80% identical to a TD26 nucleic acid. In some aspects, said nucleic acid comprises a nucleotide sequence at least 80% identical to SEQ ID NO: 14. In some aspects, said nucleic acid comprises a nucleotide sequence at least 80% identical to SEQ ID NO: 15. In some aspects, said nucleic acid comprises one or more naturally occurring single nucleotide polymorphisms.

In some embodiments, a TD26 protein of the present invention comprises an amino acid sequence at least 80% identical to a TD26 protein. In some embodiments a TD26 protein of the invention comprises an amino acid sequence at least 80% identical to the secreted portion of a TD26 protein (i.e., a portion excluding the signal sequence). In some aspects, said protein comprises an amino acid sequence at least 80% identical to SEQ ID NO: 1. In some aspects, said protein comprises an amino acid sequence at least 80% identical to SEQ ID NO: 2. In some aspects, said protein comprises an amino acid sequence at least 80% identical to SEQ ID NO: 3. In some aspects, said protein comprises an amino acid sequence at least 80% identical to SEQ ID NO: 4. In some aspects, said protein sequence comprises one or more naturally occurring amino acid variations.

TABLE 1
TD26 Single Nucleotide Polymorphisms

| dbSNP rs# | dbSNP | mRNA | Protein | Amino Acid |
|---|---|---|---|---|
| cluster id | Allele | Pos | Residue | Pos |
| rs59168178 | G/A | 32 | Ala/Thr | 5 |
| rs892066 | C/G | 46 | Leu/Leu | 9 |
| rs1541922 | T/C | 139 | His/His | 40 |
| rs142800818 | G/A | 170 | Gly/Ser | 51 |
| rs2278426 | C/T | 194 | Arg/Trp | 59 |
| rs147405465 | T/C | 240 | Ile/Thr | 74 |
| rs74810158 | C/T | 266 | Arg/Trp | 83 |
| rs145464906 | C/T | 380 | Gln/xxx | 121 |
| rs79566395 | G/A | 407 | Val/Met | 130 |
| rs75726972 | C/T | 436 | Ser/Ser | 139 |
| rs34056604 | G/A | 459 | Arg/Gln | 147 |
| rs192460764 | C/T | 533 | Arg/Trp | 172 |

A nucleic acid or polypeptide sequence will typically be from a mammal including, but not limited to, primate, e.g., human; rodent, e.g., rat, mouse, hamster; cow, pig, horse, sheep, or any mammal. Truncated forms of these referenced nucleic acids or proteins are included in the definition.

The term “polypeptide” refers, in one embodiment, to a protein or, in another embodiment, to protein fragment or fragments or, in another embodiment, to a string of amino acids. In one embodiment, reference to “peptide” or “polypeptide” is meant to include native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N-terminal, C-terminal or peptide bond modifications, including, but not limited to, backbone modifications, and residue modification. Methods for preparing peptidomimetic compounds are known in the art and are specified, for example, in Quantitative Drug Design, C.A. Ramsden Gd., Chapter 17.2, F. Choplin Pergamon Press (1992).

In other embodiments described herein, methods of the invention comprise administration of one or more nucleic acid molecules encoding TD26 proteins and polypeptides or encoding functional fragments thereof. A nucleic acid molecule encoding human TD26 polypeptide has been deposited under NCBI Reference Sequence NM_{—}018687.6 and is shown below:

(SEQ ID NO: 14)

ATGCCAGTGCCTGCTCTGTGCCTGCTCTGGGCCCTGGCAATGGTGACCC
GGCCTGCCTCAGCGGCCCCCA
TGGGCGGCCCAGAACTGGCACAGCATGAGGAGCTGACCCTGCTCTTCCA
TGGGACCCTGCAGCTGGGCCA
GGCCCTCAACGGTGTGTACAGGACCACGGAGGGACGGCTGACAAAGGCC
AGGAACAGCCTGGGTCTCTAT
GGCCGCACAATAGAACTCCTGGGGCAGGAGGTCAGCCGGGGCCGGGATG
CAGCCCAGGAACTTCGGGCAA
GCCTGTTGGAGACTCAGATGGAGGAGGATATTCTGCAGCTGCAGGCAGA
GGCCACAGCTGAGGTGCTGGG
GGAGGTGGCCCAGGCACAGAAGGTGCTACGGGACAGCGTGCAGCGGCTA
GAAGTCCAGCTGAGGAGCGCC
TGGCTGGGCCCTGCCTACCGAGAATTTGAGGTCTTAAAGGCTCACGCTG
ACAAGCAGAGCCACATCCTAT
GGGCCCTCACAGGCCACGTGCAGCGGCAGAGGCGGGAGATGGTGGCACA
GCAGCATCGGCTGCGACAGAT
CCAGGAGAGACTCCACACAGCGGCGCTCCCAGCCTGA

A nucleic acid molecule encoding mouse EG624219 polypeptide has been deposited under NCBI Reference Sequence NM_{—}001080940.1 and is shown below:

(SEQ ID NO: 15)

ATGGCTGTGCTTGCTCTCTGCCTCCTGTGGACCTTAGCATCAGCAGTGC
GACCCGCTCCAGTGGCCCCTC
TGGGTGGTCCAGAGCCAGCTCAATATGAAGAGCTGACCCTGCTCTTTCA
CGGGGCCCTGCAGCTAGGCCA
GGCCCTCAATGGCGTGTACAGAGCCACAGAGGCTCGCCTGACAGAAGCT
GGGCACAGCCTGGGCCTCTAT
GACAGAGCACTGGAATTCCTGGGGACAGAAGTCAGGCAGGGCCAGGATG
CCACACAGGAGCTTCGCACCA
GCCTGTCGGAGATTCAGGTGGAAGAGGACGCTTTACACCTTCGAGCTGA
AGCCACAGCCCGATCACTGGG
GGAAGTGGCCCGGGCCCAGCAGGCTCTGCGGGACACTGTACGGAGACTA
CAAGTGCAGCTGAGAGGCGCC
TGGCTCGGTCAAGCCCACCAAGAATTTGAGACCTTAAAGGCTCGAGCTG
ATAAGCAGAGCCACCTCTTAT
GGGCTCTCACTGGCCACGTGCAGCGACAGCAGCGGGAGATGGCAGAGCA
GCAACAGTGGCTGCGACAGAT
CCAGCAGAGACTCCACACAGCAGCCCTCCCAGCCTGA

It will be understood that, due to the degeneracy of the genetic code, other nucleotide sequences can be identified or synthesized which encode equivalent polypeptides, and these nucleotide sequences are within the scope of the invention. Moreover, the skilled artisan will readily be able to determine portions of the nucleotide sequences which encode desirable portions of TD26 polypeptides. For example, the skilled artisan will be able to identify the portion of SEQ ID NO: 14 which encodes a CCD domain of the corresponding TD26 polypeptide.

In the methods described herein which include the administration and uptake of exogenous DNA into cells (i.e., gene transduction or transfection), commonly used gene transfer methods will be known to the skilled artisan. The nucleic acids can be in the form of naked DNA or the nucleic acids can be in a vector utilized for delivering the nucleic acids to the cells, for example, retroviral vectors, adenoviral vectors, adeno-associated viral (AAV) vectors, lentiviral vectors, pseudotyped retroviral vectors. The vector can be a commercially available preparation, such as an adenovirus vector (Quantum Biotechnologies, Inc. (Laval, Quebec, Canada). As described herein, the present invention also provides a vector comprising a nucleic acid agent, either of which can be in a pharmaceutically acceptable carrier. Such nucleic acids and vectors can be used in gene therapy protocols to treat a subject in accordance with the methods of the invention.

Alternatively, the nucleic acid of this invention can be administered to the cell in a liposome. As one example, delivery can be via commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the nucleic acid or vector of this invention can be delivered in vivo by electroporation, the technology for which is available from Genetronics, Inc. (San Diego, Calif.), as well as by means of a SONOPORATION machine (ImaRx Pharmaceutical Corp., Tucson, Ariz.). The cell can be any cell which can take up and express exogenous nucleic acid; said cell may be present in vivo or ex vivo (e.g., in culture medium).

If ex vivo methods are employed, cells or tissues can be removed and maintained outside the body according to standard protocols well known in the art. The nucleic acids of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

For ex vivo therapy, vectors may be introduced into stem cells taken from the patient and clonally propagated for autologous transplant back into that same patient. Delivery by transfection and by liposome injections may be achieved using methods which are well known in the art.

In certain embodiments, the nucleic acids encoding the TD26 polypeptides of the present invention comprise a synthetic modified mRNA produced by in vitro transcription. For example, such mRNA can include, for example, from 5′ to 3′, a 5° guanine cap, a 5′ untranslated region (UTR) containing a strong Kozak sequence for translation initiation and an alpha-globin 3′ untranslated region (UTR) that terminates with a polyA tail. Cytosolic delivery of such synthetic modified mRNAs into mammalian cells for subsequent translation of the mRNA in vivo can be accomplished by electroporation or by complexing the modified mRNA with a cationic vehicle to enhance uptake by endocytosis. (Schlaeger et al., Cell Stem Cell. 7(5):618-630 (2010)).

The mode of administration of the nucleic acid or vector can vary predictably according to the disease being treated and the tissue being targeted. The nucleic acid or vector may be administered orally, parenterally (e.g., intravenously), by intramuscular injection, by intraperitoneal injection, transdermally, extracorporeally, topically or the like, although intravenous administration is typically preferred. The exact amount of the nucleic acid or vector required will vary from subject to subject, depending on the species, age, weight and general condition of the subject, the severity of the disorder being treated, the particular nucleic acid or vector used, its mode of administration and the like.

Parenteral administration of the nucleic acid or vector of the present invention, if used, is generally characterized by injection. Injectables can be prepared in conventional forms, either as liquid solutions or suspensions, solid forms suitable for solution or suspension in liquid prior to injection, or as emulsions. A more recently revised approach for parenteral administration involves use of a slow release or sustained release system such that a constant dosage is maintained.

In other embodiments, methods of increasing beta cell replication can be effected by administration of one or more insulin receptor antagonists. The present invention contemplates the use of an insulin receptor antagonist to increase beta cell replication, for example, by interfering with the ability of insulin to interact with the insulin receptor, as well as by neutralizing the biological effects of insulin. The present invention also contemplates the use of an insulin receptor antagonist that is capable of interfering with the ability of insulin to bind to the insulin receptor, as well as any agent that is capable of neutralizing the biological effects of insulin. In some embodiments the insulin receptor antagonist is capable of increasing the level or activity of TD26, preferably without markedly increasing in blood glucose levels or only doing so transiently. Insulin receptor antagonists include peptide antagonists such as S661, S961 or RB537. S661, S961 and RB537 are peptide mimetics of insulin that bind the insulin receptor but do not transmit the signal that insulin effects (see, e.g., WO2007039606, the entirety of which is incorporated herein by reference).

S661 and S961 are 43 amino acid peptides that share the amino acid sequence GSLDESFYDWFERQLGGGSGGSSLEEEWAQIQCEVWGRGCPSY (SEQ ID NO: 16). The C-terminus of S661 is an amide, whereas the C-terminus of S961 is an acid. RB537 has the amino acid sequence: MADYKDDDDKGSLDESFYDWFERQLGGGSGGSWEDQEWAWVQCEVYGRGCPSAAAGAPV PYPDPLEPRPG (SEQ IS NO: 17). In certain embodiments, a functional portion of RB537 includes at least an affinity-optimized first peptide having the amino acid sequence GSLDESFYDWFERQLG (SEQ ID NO: 18) linked via a 6 amino acid sequence GGSGGS (SEQ ID NO: 19) to an affinity-optimized second peptide having the amino acid sequence WLDQEWAWVQCEVYGRGCPS (SEQ ID NO: 20). In such embodiments, the functional portion of RB537 can further include a first peptide-flanking epitope tag (e.g., FLAG (DYKDDDDK) (SEQ ID NO: 21)) at the N-terminus and a second peptide-flanking epitope E-Tag (GAPVPYPDPLEPR) (SEQ ID NO: 22) at the C-terminus.

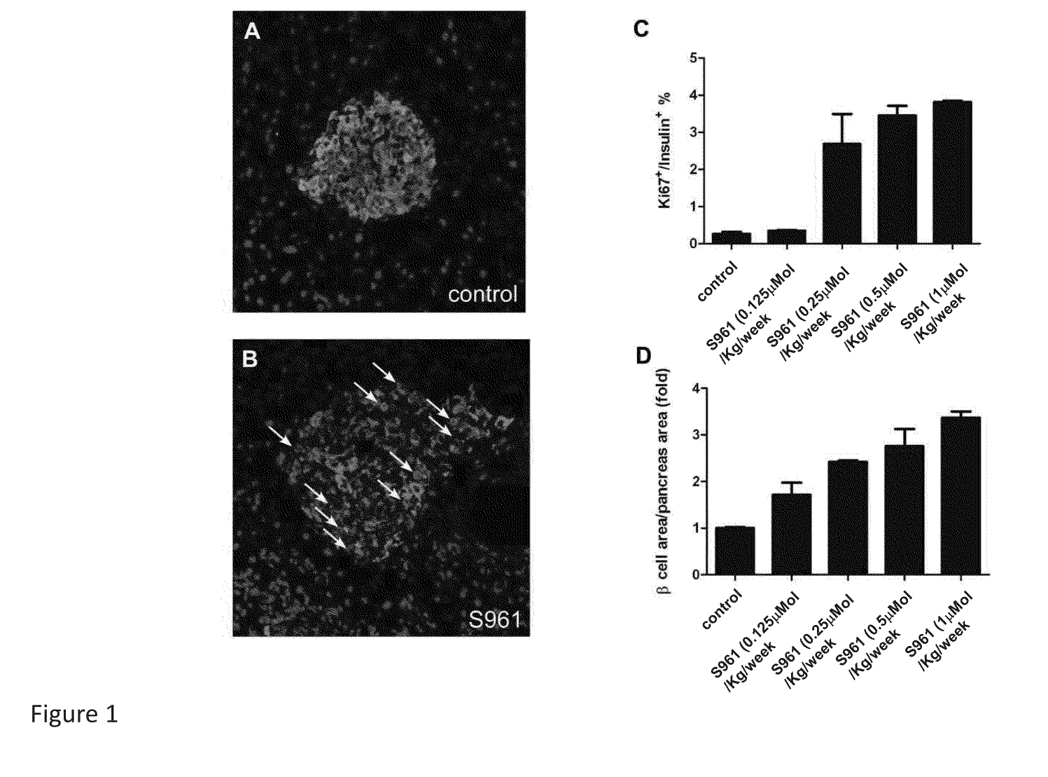

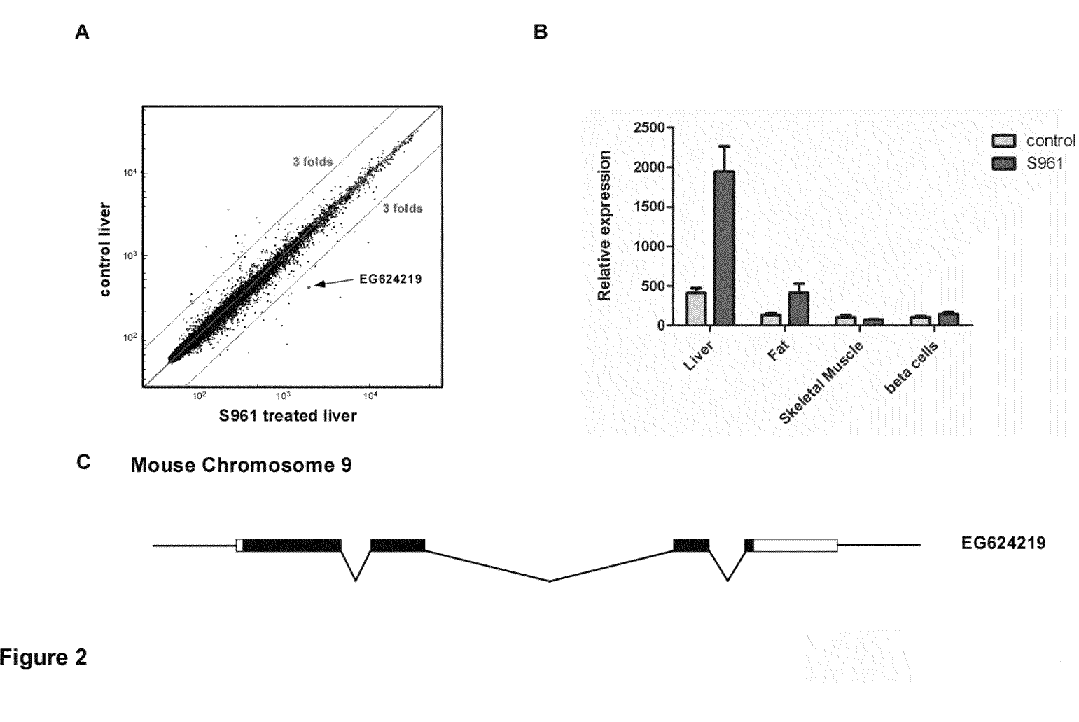

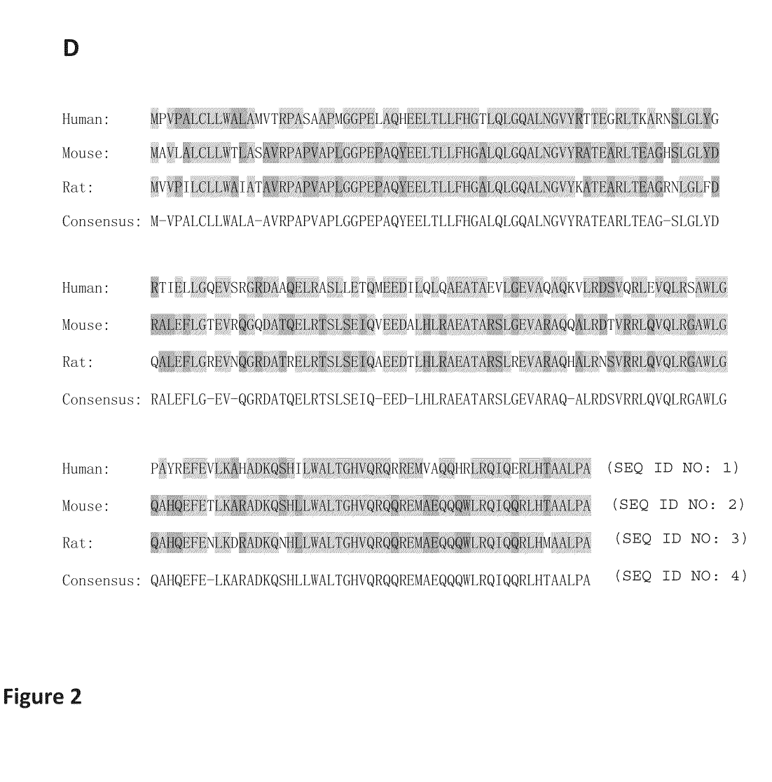

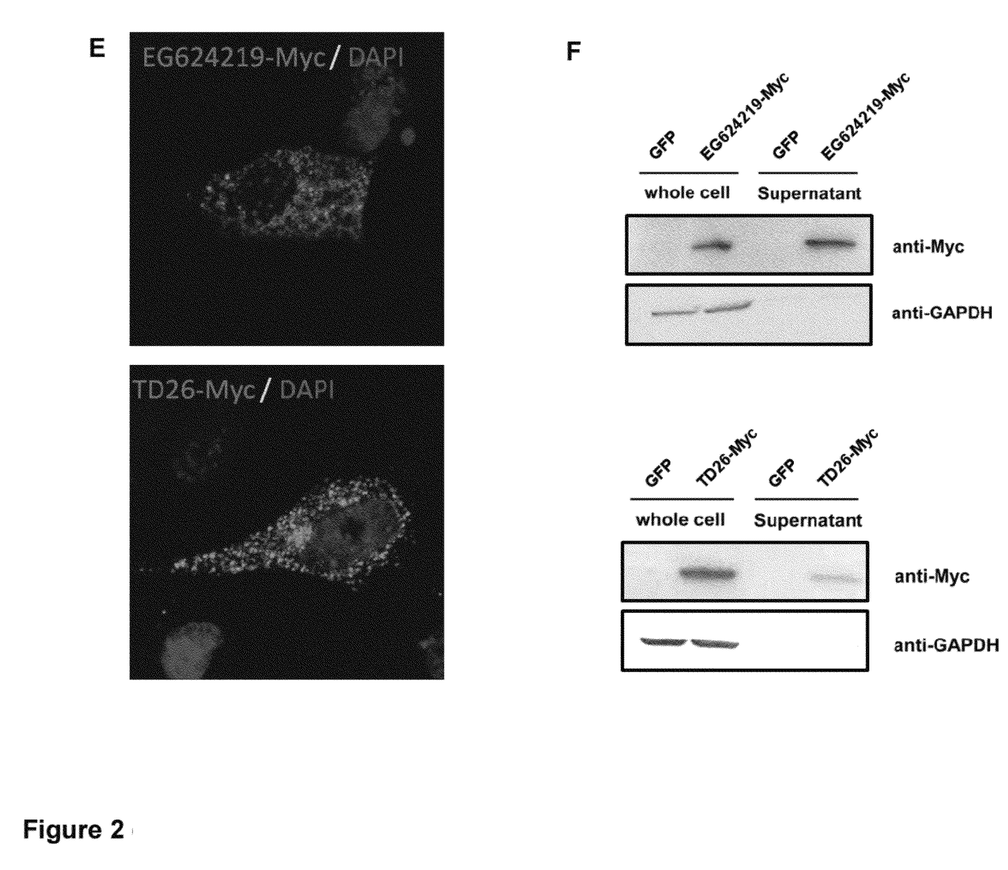

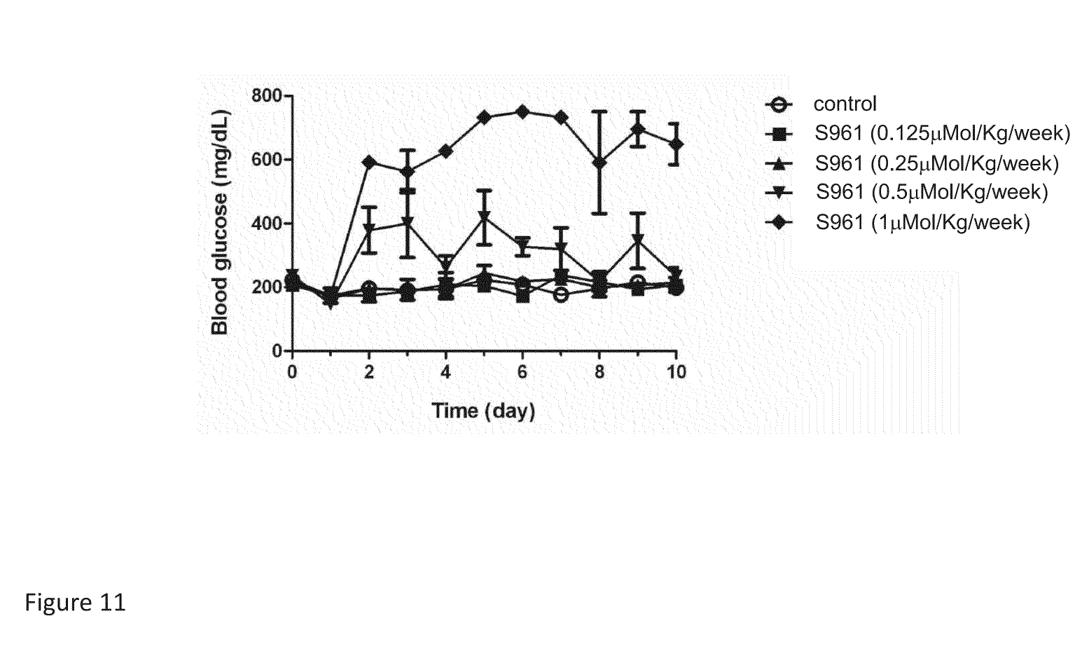

Previous reports have demonstrated that upon administration of peptide insulin receptor antagonist S661 to obese rats, a marked increase in blood glucose levels was observed (Schäffer et al., Biochem Biophys Res Commun. 376(2):380-383 (2008)). In contrast, work described herein demonstrates that at low doses, S961 does not increase blood glucose levels in mammals. With increasing doses of S961, however, blood glucose levels increase, and the animal becomes hyperglycemic (FIG. 11). Work described herein shows that at low doses of peptide S961, TD26 expression is induced (see FIG. 2B), and beta cell replication is increased (see FIGS. 1C and 1D). As used herein, low doses of S961 will typically be less than 1 μMol/Kg/week, preferably from 0.125 μMol/Kg/week to 0.5 μMol/Kg/week. However it will be understood that increased doses may be useful as well, particularly if utilized in conjunction with an anti-hyperglycemic agent.

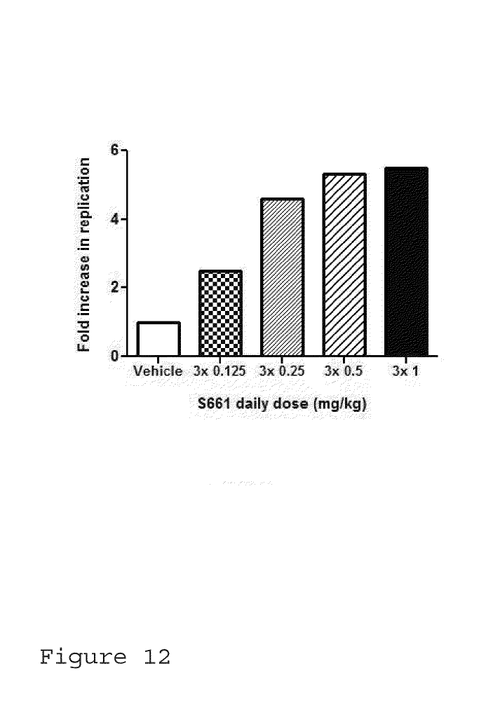

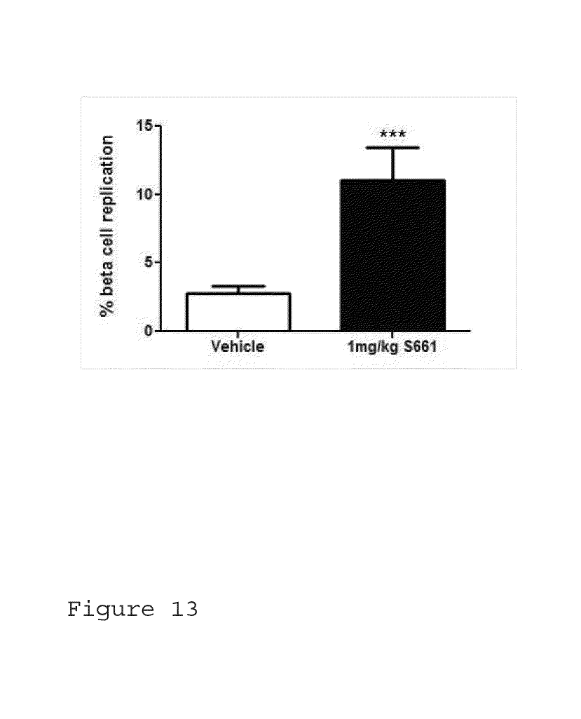

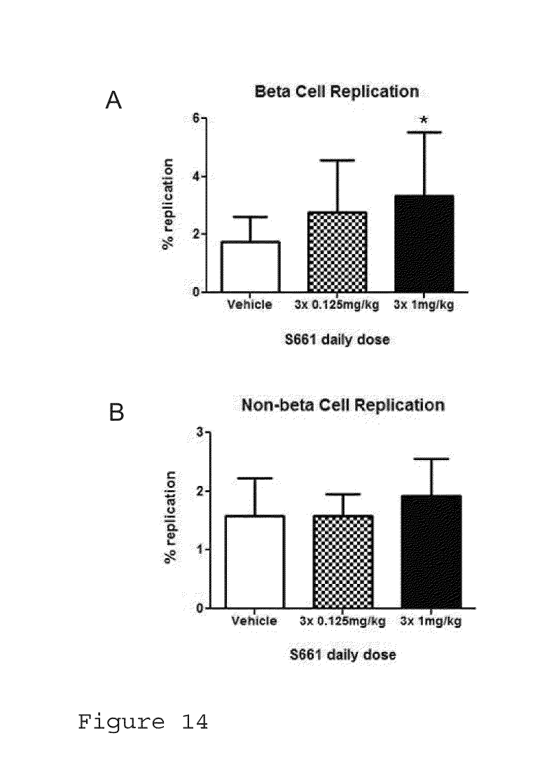

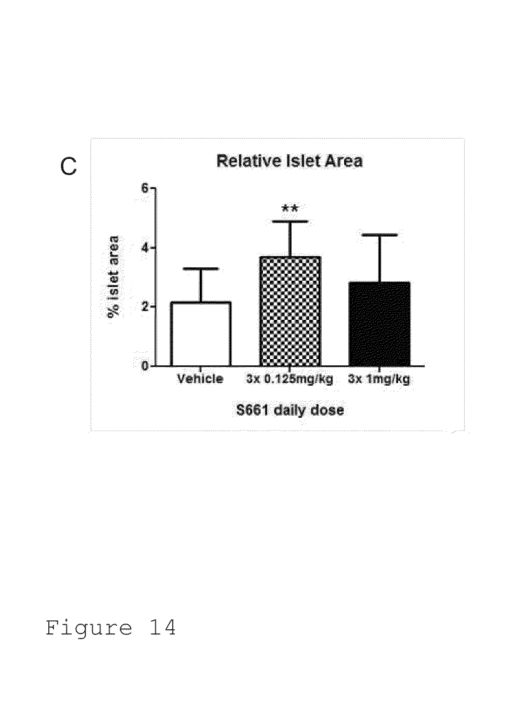

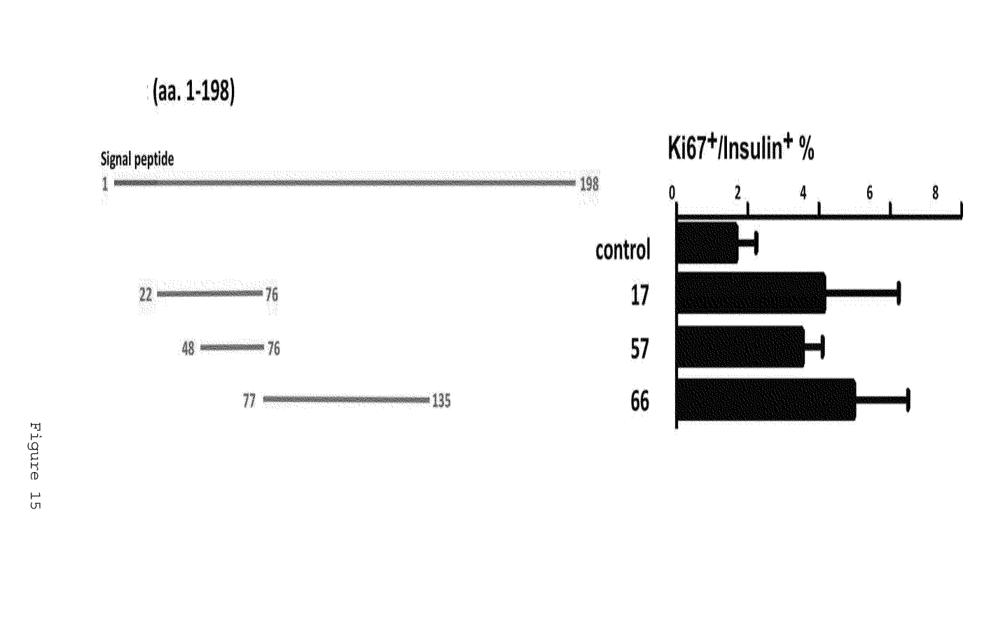

Moreover, work described herein shows that upon administration of peptide S661, beta cell replication is increased in normal individuals (see FIGS. 12 and 13) and in a model of human type 2 diabetes (see FIG. 14A). Work described herein also shows that upon administration of peptide S661, islet area increases relative to total pancreas area (FIG. 14C).

Insulin receptor antagonists may be administered either as a monotherapy or as a combination therapy with other pharmaceutical agents. For example, they may be administered together with other pharmaceutical agents suitable for the treatment or prevention of diabetes and/or obesity and/or metabolic syndrome. In some embodiments, a combination therapy includes co-administration of an insulin receptor antagonist and an additional agent. As used herein, the term “co-administration” refers to administration of two or more biologically active substances to a subject. Co-administration can be simultaneous or sequential. The two or more biologically active substances can be part of a single composition or separate compositions. In some embodiments, a combination therapy of the present invention comprises co-administration of an insulin receptor antagonist with one or more blood glucose lowering agents or agents that are beneficial to beta cells. These agents include, but are not limited to, Metformin or other Biguanides, DPP4 inhibitors, Sulfonylureas or Metiglitinides, SGLT2 inhibitors, Glucokinase activators, Thiazolidinediones, PPARdelta agonists, non-activating PPARgamma modulators, Glp-1 analogs, GIP analogs, Glp-1-receptor agonists, combined Glp-1/GIP receptor agonists, FGF21, agonistic FGFR monoclonal antibodies, Oxyntomodulin analogs, IAPP analogs, Leptin or Leptin analogs, Adiponectin or Adiponectin analogs, Insulin or Insulin analogs, proton pump inhibitors or gastrin receptor agonists, Reg family proteins/Reg family protein derived peptides or alpha-glucosidase inhibitors. Further, they may be administered together with pharmaceutical agents which have an immunosuppressive or immunomodulatory activity, e.g., antibodies, polypeptides and/or peptidic or non-peptidic low molecular weight substances.

Compounds that decrease TD26 levels or activity include, for example, TD26 antibodies or fragments thereof or a nucleic acid that is complementary to a nucleic acid encoding a TD26 polypeptide (e.g., antisense oligonucleotides, ribozymes or siRNA). Production of suitable antibodies is well known in the art, and may comprise methods as described in, for example, Harlow and Lane, Antibodies: A Laboratory Manual, Cold Spring Harbor Laboratory, New York, 1988.

“siRNA” is a double stranded RNA molecule which prevents translation of a target mRNA. Standard techniques of introducing siRNA into a cell are used, including those in which DNA is a template from which an siRNA is transcribed. The siRNA includes a sense TD26 nucleic acid sequence, an antisense TD26 nucleic acid sequence or both. Optionally, the siRNA is constructed such that a single transcript has both the sense and complementary antisense sequences from the target gene, e.g., a hairpin.

Binding of the siRNA to a TD26 transcript in the target cell results in a reduction in TD26 production by the cell. The length of the oligonucleotide is typically at least about 10 nucleotides and may be as long as the naturally-occurring TD26 transcript. Preferably, the oligonucleotide is 19-25 nucleotides in length. Most preferably, the oligonucleotide is less than 75, 50, 25 nucleotides in length.

Agents described herein for use in the described methods (also referred to herein as “active compounds”) can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

TD26 nucleic acids and polypeptide and effectors/modulators thereof may be administered either as a monotherapy or as a combination therapy with other pharmaceutical agents. For example, they may be administered together with other pharmaceutical agents suitable for the treatment or prevention of diabetes and/or obesity and/or metabolic syndrome. In some embodiments, a combination therapy includes co-administration of TD26 and an additional agent. As used herein, the term “co-administration” refers to administration of two or more biologically active substances to a subject. Co-administration can be simultaneous or sequential. The two or more biologically active substances can be part of a single composition or separate compositions. In some embodiments, a combination therapy of the present invention comprises co-administration of a TD26 polypeptide with one or more blood glucose lowering agents or agents that are beneficial to beta cells. These agents include, but are not limited to, Metformin or other Biguanides, DPP4 inhibitors, Sulfonylureas or Metiglitinides, SGLT2 inhibitors, Glucokinase activators, Thiazolidinediones, PPARdelta agonists, non-activating PPARgamma modulators, Glp-1 analogs, GIP analogs, Glp-1-receptor agonists, combined Glp-1/GIP receptor agonists, FGF21, agonistic FGFR monoclonal antibodies, Oxyntomodulin analogs, IAPP analogs, Leptin or Leptin analogs, Adiponectin or Adiponectin analogs, Insulin or Insulin analogs, proton pump inhibitors or gastrin receptor agonists, Reg family proteins/Reg family protein derived peptides or alpha-glucosidase inhibitors. Further, they may be administered together with pharmaceutical agents which have an immunosuppressive activity, e.g., antibodies, polypeptides and/or peptidic or non-peptidic low molecular weight substances.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition should be sterile and should be fluid to the extent that easy syringeability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, or polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze drying that yields a powder of the active ingredient plus any additional desired ingredient from a previously sterile filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811, incorporated fully herein by reference.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. The pharmaceutical compositions and agents described herein can be included in a container, pack, or dispenser together with instructions for administration.

The present invention provides for both prophylactic and therapeutic methods of treating a subject at risk of (or susceptible to) a disorder or having a disorder associated with pancreatic beta cell degeneration, aberrant insulin production and/or blood glucose levels. As used herein the term “pancreatic beta cell degeneration” is intended to mean loss of beta cell function (particularly insulin production and/or secretion), beta cell dysfunction, and death of beta cells, such as necrosis or apoptosis of beta cells.

As used herein, the term “treatment” is defined as the application or administration of a therapeutic agent to a patient, or application or administration of a therapeutic agent to an isolated tissue or cell line from a patient, said patient having a disease, a symptom of disease or a predisposition toward a disease, with the purpose to cure, heal, alleviate, relieve, alter, remedy, ameliorate, improve or affect the disease, the symptoms of disease or the predisposition toward disease. Thus, treating may include suppressing, inhibiting, preventing, treating, or a combination thereof. Treating refers, inter alia, to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers, inter alia, to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. In one embodiment the symptoms are primary, while in another embodiment symptoms are secondary. “Primary” refers to a symptom that is a direct result of a disorder, e.g., diabetes, while, secondary refers to a symptom that is derived from or consequent to a primary cause. Symptoms may be any manifestation of a disease or pathological condition.

As described herein, pancreatic beta cell mass can be increased by administering to an animal (e.g., a human) a compound that increases TD26 level or activity in a tissue or cell (e.g., TD26 protein). As used herein, the terms “beta cell proliferation” and “beta cell replication” are used interchangeably. Increased beta cell mass occurs via increased proliferation or replication of beta cells, enhanced differentiation of precursor cells to a beta cell lineage, and/or or diminished beta cell turnover or death. The increase in β-cell mass can be at least 5%, 10%, 20%, 30%, 40%, 50%, 50%, 70%, 80%, 90%, 1-fold, 2-fold, 5-fold, 10-fold, 50-fold, 100-fold or more compared to the β-cell mass prior to onset of treatment.

As used herein, “increasing β-cell replication” means that β-cells replicate at a faster rate and/or more frequently. In some embodiments of this and other aspects of the invention, β-cell replication is increased by at least 5%, 10%, 20%, 30%, 40%, 50%, 50%, 70%, 80%, 90%, 1-fold, 1.1-fold, 1.5-fold, 2-fold, 3-fold, 4-fold, 5-fold, 10-fold, 50-fold, 100-fold or more higher relative to an untreated control. The % or fold increase in β-cell replication can be determined by measuring number of replicating β-cells during or after treatment with a compound described herein relative to a control. Increase in replication can also be based on ratios of replicating cells to total number of cells in the respective treated and untreated control. In some embodiments, total numbers of cells in the treated and untreated controls are used to determine the replication frequency.

In some embodiments, “increasing β-cell replication” also includes an increase in β-cell number due to differentiation of β-cell progenitors into β-cells. In an alternative embodiment, “increasing β-cell replication” does not include an increase in 3-cell number due to differentiation of β-cell progenitors into β-cells.

For ex vivo methods of the invention, increased β-cell replication can be monitored by any method known in the art for measuring cell replication. For example, β-cell replication can be determined by measuring the expression of at least one cell replication marker, e.g., Ki-67 or PH3. A non-limiting example is the quantitative immunofluorescent assay that measures mitotic index by monitoring histone H3 phosphorylation on serine 10 (H3-P), a mitosis-specific event (Ajiro et al., J. Biol. Chem. 271:13197-201. 1996; Goto et al, J Biol Chem. 274:25543-9, 1999). Increase in β-cell replication can also be based on an increase in the total number of β-cells in the treated versus untreated control. In some instances, increased β-cell replication can be based on the ratio of β-cells to total cells for the treated and untreated controls. β-cell replication can be measured by monitoring the number of cells co-expressing Ki-67 and/or PH3, and PDX-1.

For in vivo methods of the invention, increased β-cell replication can be evaluated indirectly by measuring blood insulin levels. Without wishing to be bound by theory, blood insulin level is an indirect measure of the number of β-cells, e.g., β-cell mass in the subject. Therefore, blood insulin levels before and after onset of treatment can indirectly provide a relative measure of number of β-cells in the subject before and after onset of treatment. β-cell mass in a subject can also be determined by measuring the fasting blood glucose concentration in the subject. A curvilinear relationship between β-cell mass and fasting blood glucose concentrations in humans is disclosed in Ritzel, et. al., Diabetes Care (2006), 29:717-718, contents of which are herein incorporated by reference in their entirety. Alternatively, in vivo uptake of radioligand [11C]DTBZ (dihydrotetrabenazine), which specifically binds to VMAT2, by β-cells can be measured by positron emission tomography (P.E.T.) scanning. This radioligand has been used previously in human subjects in clinical trials evaluating P.E.T scanning of the brain in patients with bipolar illness and schizophrenia compared to healthy control subjects. U.S. Pat. Pub. No. 2009/0202428 describes use of DTBZ for imaging endocrine pancreas β-cell mass in type 1 diabetes, the contents of which are herein incorporated by reference in theory entirety.

Methods for estimating in vivo (3-cell mass are also described in, for example, Antkowiak, P. F., et al., Am J Physiol Endocrinol Metab (2009), 296:E573-E5788; Bergman, R. N., et al., Am J Physiol (1979), 236: E667-E677; Brunzell J. D., et al., J. Clin. Endocrinol. Metab (1976), 42:222-229; DeFronzo, R. A., et al., Am J Physiol (1979), 237: E214-E223; Evgenov N. V., et al., Nat Med (2006), 12:144-148; Kjems, L. L., et al., Diabetes (2001), 50: 2001-2012; Larsen, M. O., et al., Diabetologia (2003), 46: 195-202; Larsen, M. O., et al., Diabetes (2003), 52: 118-123; Larsen, M. O. et al., Am J Physiol Endocrinol Metab (2005), 2006, 290: E670-E677; McCulloch, D. K., et al., Diabetes (1991), 40: 673-679; Meier, J. J., et al. Diabetes, (2009), 58: 1595-1603; Souza F, et al., J. Clin. Invest. (2006), 116: 1506-1513; Tobin B. W., et al., Diabetes (1993), 42:98-105; and Ward, W. K., et al., J Clin Invest (1984), 74: 1318-1328, the contents of which are herein incorporated by reference in their entirety.

For in vivo methods, a therapeutically effective amount of a compound described herein can be administered to a subject. Methods of administering compounds to a subject are known in the art and easily available to one of skill in the art. Examples of such routes include parenteral, enteral, and topical administration. Parenteral administration is usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. Administration can be systemic administration, or localized, as determined necessary by the skilled practitioner.

Cell proliferation (e.g., beta cell proliferation) may be determined via any number of methods well known in the art and as exemplified herein, for example via measuring uptake of a labeled substrate, such as tritiated thymidine. Tissues and cells may be in direct contact with agents and compositions of the invention, or exposed indirectly, through methods well described in the art. For example, cells can be grown in culture media in vitro, wherein the media is supplemented with polypeptides, nucleic acids, vectors or other agents described herein. In one embodiment, the cells being contacted are primary cultures. In one embodiment, “primary culture” denotes a mixed cell population that permits interaction of many different cell types isolated from a tissue. In another embodiment, a primary culture may be a purified cell population isolated from a tissue. In one embodiment, the primary culture may be enriched for a particular population. In one embodiment, enrichment may comprise cell sorting via means well known in the art, such as, for example fluorescent activated cell sorting (FACS), for cell populations, for example, expressing a particular cell surface marker, or in another embodiment, lacking cell surface expression of a particular marker.

Alternatively, contacting a cell may include any route of administration to a subject, for example, oral or parenteral administration of a polypeptide, peptide, nucleic acid, vector or composition of this invention to a subject, wherein administration results in in vivo cellular exposure to these materials, within specific sites within a body. In some embodiments the methods comprise a step of administering the contacted cell to the subject, such as, for example, ex vivo cellular therapy. In some embodiments the cells administered to the subject are autologous or in another embodiment, allogenic with respect to the subject.

As further described herein, blood insulin concentration is increased by administering to a subject an agent that increases TD26 level or activity. Moreover, blood glucose levels can be decreased by administering to a subject a compound that increases TD26 level or activity. Preferably, blood glucose levels decrease to normal levels, i.e., to blood glucose levels of a healthy individual without a disease.

In certain embodiments, the subject is a human subject or patient. In particular embodiments the subject is suffering from or is susceptible to developing a disorder associated with aberrant insulin production or responsiveness or aberrant blood glucose levels. Disorders include, but are not limited to, diabetes (e.g., Type I or Type II), gestational diabetes, prediabetes, obesity, hyperglycemia, glucose intolerance, insulin resistance, hyperinsulinemia, metabolic syndrome, or syndrome X. The term “diabetes” refers to a disease of a mammalian subject, and includes Type 1 NIDDM-transient, Type 1 IDDM, Type 2 IDDM-transient Type 2 NIDDM, or iii another embodiment, MODY.

Subjects suffering from or at risk of such disorder are identified by methods known in the art. For example diabetes can be diagnosed by art-recognized diagnosis and treatment recommendations, e.g., from the American Diabetes Association. Obesity is diagnosed for example, by body mass index. Body mass index (BMI) is measured (kg/m2 (or lb/in2×704.5)). Alternatively, waist circumference (estimates fat distribution), waist-to-hip ratio (estimates fat distribution), skinfold thickness (if measured at several sites, estimates fat distribution), or bioimpedance (based on principle that lean mass conducts current better than fat mass (i.e., fat mass impedes current), estimates % fat) is measured. The parameters for normal, overweight, or obese individuals is as follows: Underweight: BMI <18.5; Normal: BMI 18.5 to 24.9; Overweight: BMI=25 to 29.9. Overweight individuals are characterized as having a waist circumference of >94 cm for men or >80 cm for women and waist to hip ratios of >0.95 in men and >0.80 in women. Obese individuals are characterized as having a BMI of 30 to 34.9, being greater than 20% above “normal” weight for height, having a body fat percentage >30% for women and 25% for men, and having a waist circumference >102 cm (40 inches) for men or 88 cm (35 inches) for women. Individuals with severe or morbid obesity are characterized as having a BMI of >35.

Efficacy of treatment is determined in association with any known method for diagnosing the disorder. Alleviation of one or more symptoms of the disorder indicates that the compound confers a clinical benefit. Any of the therapeutic methods described to above may be applied to any suitable subject including, for example, mammals such as dogs, cats, cows, horses, rabbits, monkeys, and most preferably, humans.

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%. In another embodiment, symptoms are alleviated such that the condition of the patient is close or equal to normal humans not suffering from the condition.

Treatment of Diabetes is determined by standard medical methods. A goal of Diabetes treatment is to bring sugar levels down to as close to normal as is safely possible. Commonly set goals are 80-120 milligrams per deciliter (mg/dl) before meals and 100-140 mg/dl at bedtime. Treatment goals may also be defined via HbAlc levels. A particular physician may set different targets for the patient, depending on other factors, such as how often the patient has low blood sugar reactions. Useful medical tests include tests on the patient's blood and urine to determine blood sugar level, tests for glycosylated hemoglobin level (HbAlc; a measure of average blood glucose levels over the past 2-3 months, normal range being 4-6%), tests for cholesterol and fat levels, and tests for urine protein level. Such tests are standard tests known to those of skill in the art (see, for example, American Diabetes Association, 2011). A successful treatment program can also be determined by having fewer patients in the program with complications relating to Diabetes, such as diseases of the eye, kidney disease, or nerve disease.

The methods described herein may lead to a reduction in the severity or the alleviation of one or more symptoms of the disorder. Symptoms of diabetes include, for example, elevated fasting blood glucose levels, blood pressure at or above 140/90 mm/Hg; abnormal blood fat levels, such as high-density lipoproteins (HDL) less than or equal to 35 mg/dL, or triglycerides greater than or equal to 250 mg/dL (mg/dL=milligrams per deciliter of blood). Other symptoms of diabetes include for example frequent urination, excessive thirst, extreme hunger, unusual weight loss, increased fatigue, irritability, or blurry vision.

Delaying the onset of diabetes in a subject refers to delay of onset of at least one symptom of diabetes, e.g., hyperglycemia, hypoinsulinemia, diabetic retinopathy, diabetic nephropathy, blindness, memory loss, renal failure, cardiovascular disease (including coronary artery disease, peripheral artery disease, cerebrovascular disease, atherosclerosis, and hypertension), neuropathy, autonomic dysfunction, hyperglycemic hyperosmolar coma, or combinations thereof, for at least 1 week, at least 2 weeks, at least 1 month, at least 2 months, at least 6 months, at least 1 year, at least 2 years, at least 5 years, at least 10 years, at least 20 years, at least 30 years, at least 40 years or more, and can include the entire lifespan of the subject.

The invention also provides methods of identifying a candidate therapeutic agent for treatment of a disorder associated with a reduced level of endogenous insulin or with insulin resistance comprising contacting a suitable cell with a test agent; and determining the effect of said test agent on level or activity of TD26, wherein a test agent which increases TD26 level or activity is a candidate therapeutic agent for treatment of a disorder associated with a reduced level of endogenous insulin. In certain embodiments, the invention provides a method of identifying a candidate therapeutic agent for treatment of a disorder associated with a reduced level of endogenous insulin comprising contacting a suitable cell with a test agent; and determining the effect of said test agent on level or activity of TD26, wherein a test agent which increases TD26 level or activity is a candidate therapeutic agent for treatment of a disorder associated with a reduced level of endogenous insulin. In certain embodiments, the invention provides a method of identifying a candidate therapeutic agent for treating or preventing a disorder associated with resistance to endogenous insulin comprising contacting a suitable cell with a test agent; and determining the effect of said test agent on level or activity of TD26, wherein a test agent which increases TD26 level or activity is a candidate therapeutic agent for treating or preventing a disorder associated with resistance to endogenous insulin. In some aspects the effect of said test agent on level or activity of TD26 is assessed by determining the effect of said test agent on gene expression level of TD26. For example, gene expression can be assessed using a variety of methods known in the art, including PCR and microarray analysis. Candidate therapeutic agents can be further assessed using additional methods tailored to specific functional effects if desired.

The present invention also contemplates methods of diagnosing TD26-related disorders in an individual comprising determining TD26 levels in a sample obtained from an individual suspected of suffering from a TD26-related disorder.

The determination of TD26 levels in a sample obtained from an individual can be used to determine how to care for the TD26-related disorder in the individual. For example, since reduced or decreased TD26 levels are associated with decreased beta cell proliferation, reduced endogenous insulin production, and/or resistance to endogenous insulin, e.g., diabetes, a health-care provider can use the information pertaining to TD26 levels to assist in decisions relating to treatment of an individual.

The level of TD26 which is indicative of a TD26-related condition may be defined as the decreased level present in samples from individuals known to have a TD26-related disorder over the TD26 level in samples from individuals known to be free of a TD26-related disorder. The level of TD26 may be, for example, at least 1.1 fold, 1.2 fold, 1.3 fold, 1.4 fold, 1.5 fold, 1.6 fold, 1.7 fold, 1.8 fold, 1.9 fold, 2.0 fold, 2.1 fold, 2.2 fold, 2.3 fold, 2.4 fold, 2.5 fold, 2.6 fold, 2.7 fold, 2.8 fold, 2.9 fold, 3.0 fold, 3.1 fold, 3.2 fold, 3.3 fold, 3.4 fold, 3.5 fold, 3.6 fold, 3.7 fold, 3.8 fold, 3.9 fold, 4.0 fold, 4.1 fold, 4.2 fold, 4.3 fold, 4.4 fold, 4.5 fold, 4.6 fold, 4.7 fold, 4.8 fold, 4.9 fold, 5.0 fold, 5.1 fold, 5.2 fold, 5.3 fold, 5.4 fold, 5.5 fold, 5.6 fold, 5.7 fold, 5.8 fold, 5.9 fold, 6.0 fold, 10 fold, 15 fold, 20 fold, 50 fold or 100 fold lower in a sample from an individual with a TD26-related disorder.

The TD26 protein is detected and/or quantified in the sample using any of a number of well recognized immunological binding assays (see, e.g., U.S. Pat. Nos. 4,366,241; 4,376,110; 4,517,288; and 4,837,168). For a review of general immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

In some embodiments, the TD26 protein in the sample can also be detected and quantified using immunoblot (Western blot) analysis. Immunoblotting generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with antibodies that specifically bind the TD26 protein. The anti-TD26 antibodies specifically bind to TD26 on the solid support. These antibodies may be directly labeled or alternatively may be subsequently detected using labeled antibodies (e.g., labeled sheep anti-mouse antibodies) that specifically bind to the anti-TD26 antibody.

In some embodiments, quantitative assays of TD26 are deemed to show a positive result, e.g., elevated or decreased TD26 level, when the measured TD26 protein level is greater or less than the level measured or known for a control sample (e.g. either a level known or measured for a normal healthy individual or a “baseline/reference” level determined at a different time for the same individual. In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

In an embodiment, a method of diagnosing a TD26-related disorder in a test individual comprises

determining a TD26 level in a sample obtained from said test individual, wherein a TD26 level that is decreased in said test individual compared to a TD26 level in a normal individual is indicative of a TD26-related disorder.

In an embodiment, a method of diagnosing a TD26-related disorder in an individual comprises detecting TD26 levels in a sample from said individual, wherein TD26 level that is decreased compared to a previous TD26 level in said individual is indicative of a TD26-related disorder.

In some aspects, said TD-26 related disorder is characterized by one or more of decreased beta cell proliferation, reduced levels of endogenous insulin, and reduced sensitivity to endogenous insulin. In some aspects, said TD-26 related disorder is Type 1 or Type 2 diabetes.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice of this disclosure, suitable methods and materials are described below.

### EXAMPLES

### Example 1

### Identification of Genes Involved in Beta cell Replication

As described herein, administration of a low dose of insulin receptor antagonist S961 to a mammal produces an increase in beta cell replication (FIG. 1). Following injection of S961, gene expression in liver, muscle, and fat was analyzed because these tissues are involved in carbohydrate storage and metabolism. Of particular interest as a result of this analysis was mouse gene EG624219.

### Example 2

### Sequencing and Characterization of TD26

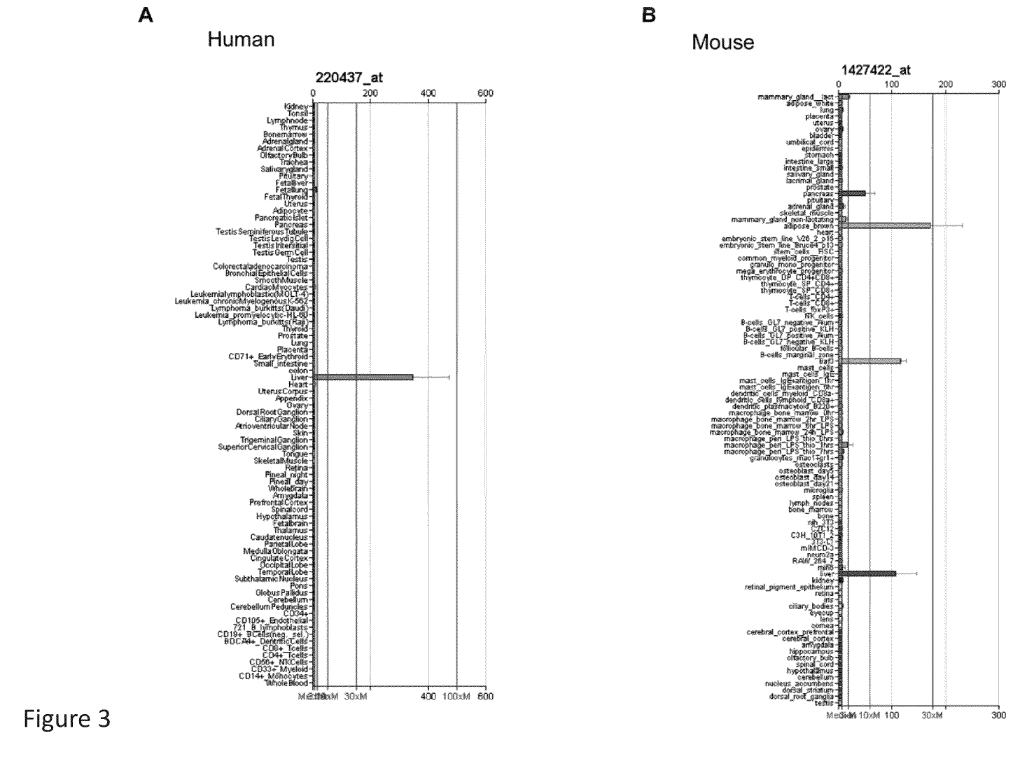

A search of the protein sequence databases revealed that the name of the human ortholog to mouse EG624219 is hepatocellular carcinoma-associated protein TD26. FIG. 2 shows sequence information for mouse gene EG624219 and human TD26. Note that the sequence predicts a signal peptide, indicating that TD26 is a secreted protein. The results of a search of publicly available databases from experiments in which mRNA abundance is measured using transcriptional arrays are shown in FIG. 3. Note the unusually specific expression of TD26 in human samples.

### Example 3

### Functional Test for TD26 in Injected Mice

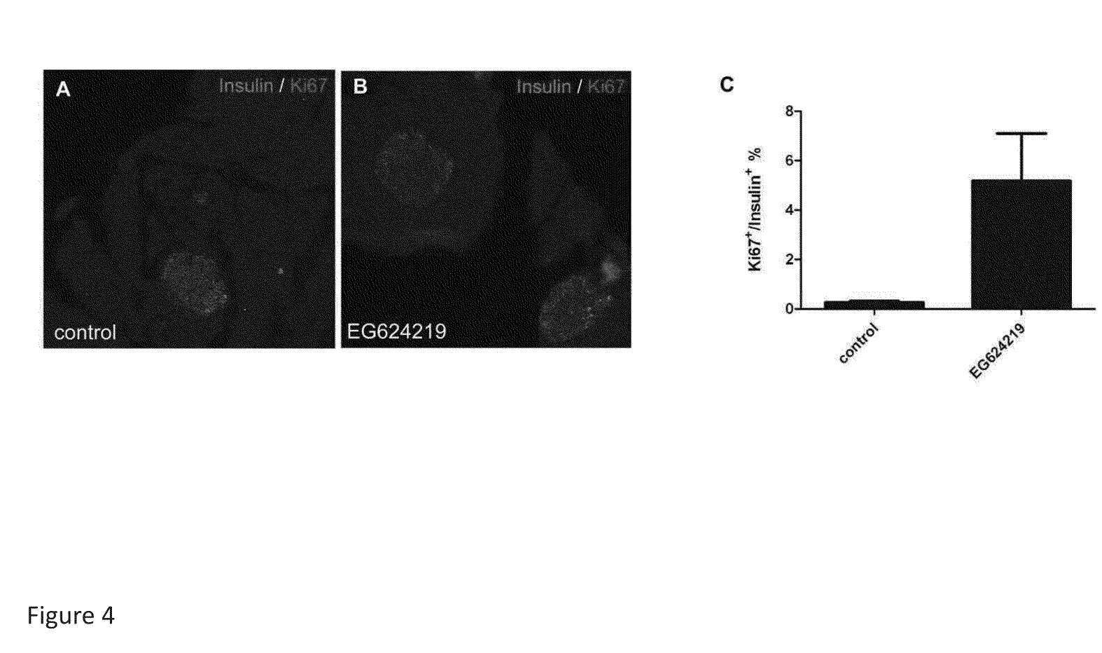

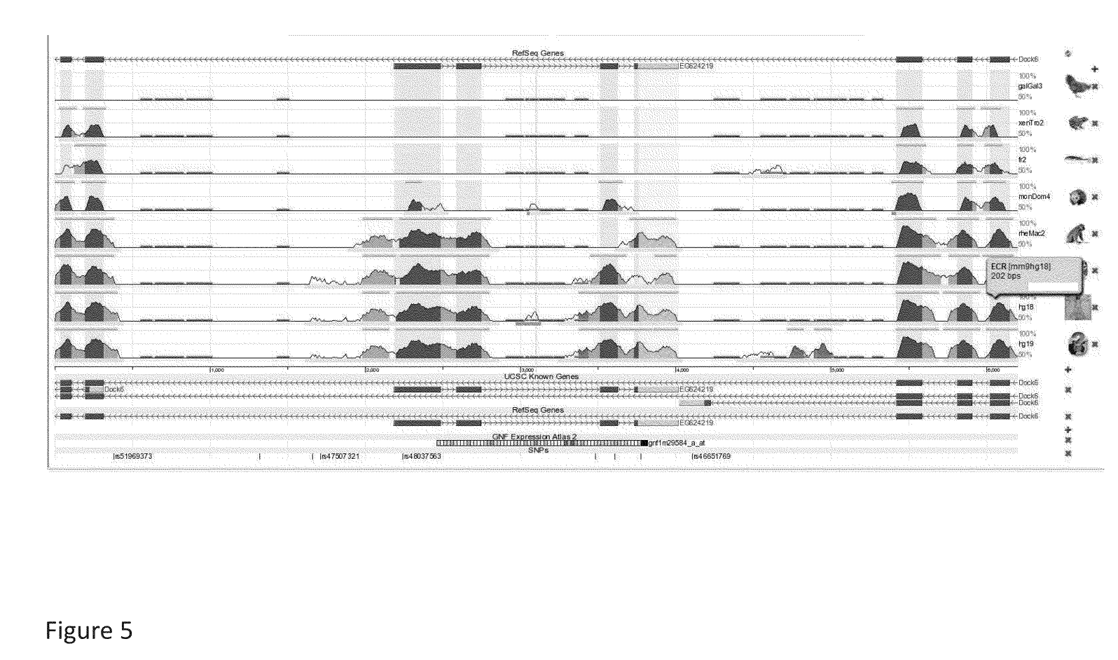

Mice were injected via tail vein with plasmid DNA containing a strong promoter driving the expression of a cDNA encoding EG624219 protein. Tail vein injection of DNA in mice causes the DNA to be expressed in liver cells (Rossmanith et al., DNA and Cell Biology 2/(11):847-853 (2002)). Liver expression was confirmed with controls showing green fluorescent protein (GFP) in liver following injection of DNA encoding GFP. The result of injecting DNA encoding EG624219 into the tail veins of mice is shown in FIG. 4. Injection of DNA encoding EG624219, but not the GFP control, causes a sharp and significant replication of beta cells. EG624319 appears to be a gene having orthologs found in mammals, for example human, rat, and mice, but not other vertebrates (FIG. 5). A preliminary sequence analysis fails to provide evidence for TD26 orthologs in chicks, frogs, fish, and other non-mammalian species.

### Example 4

### Identification of Orthologs

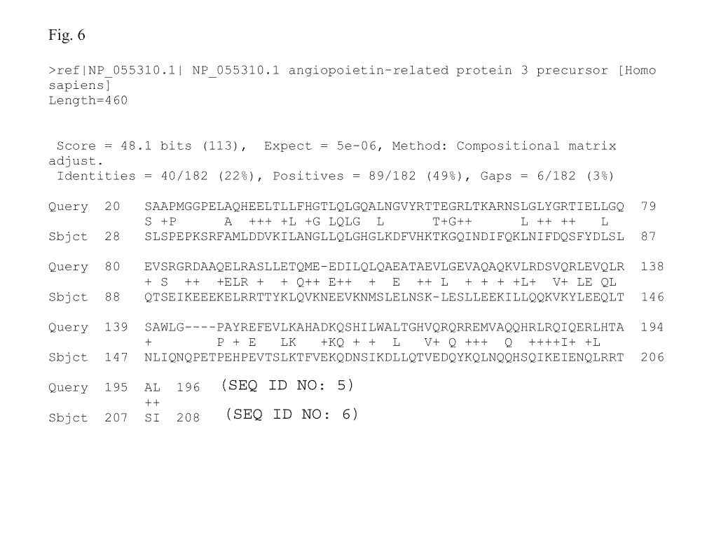

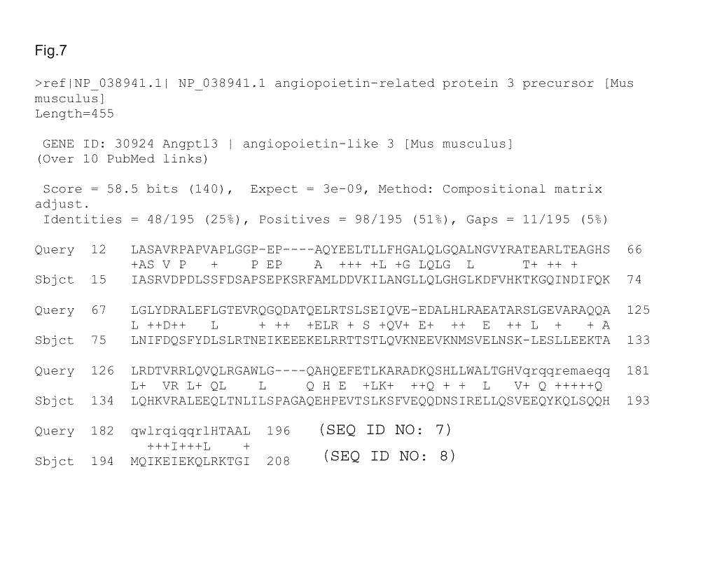

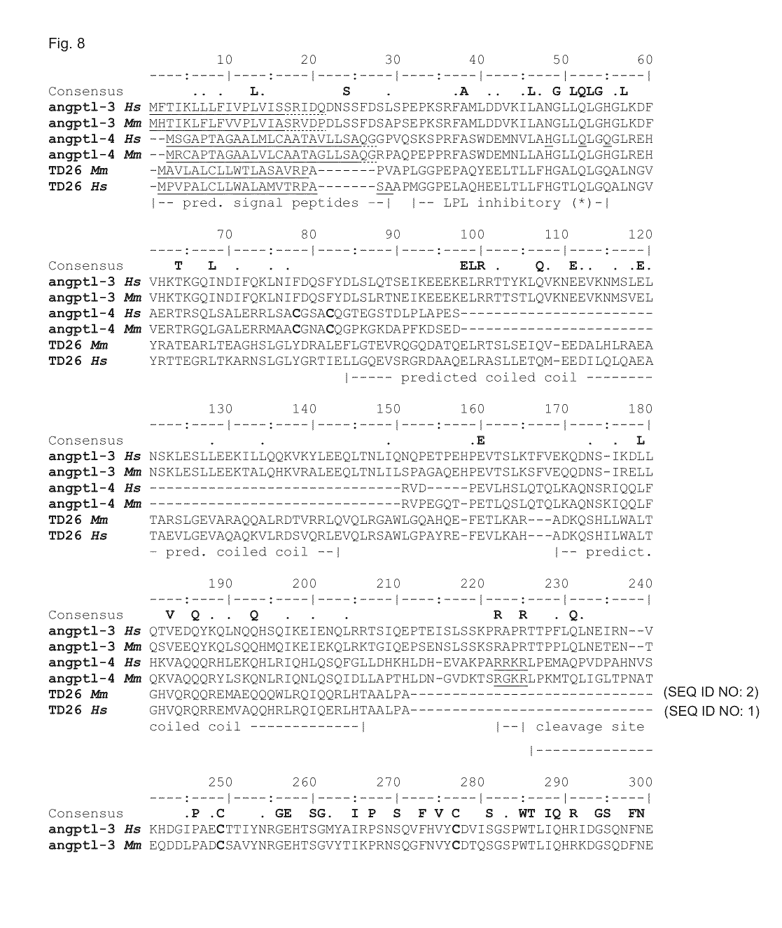

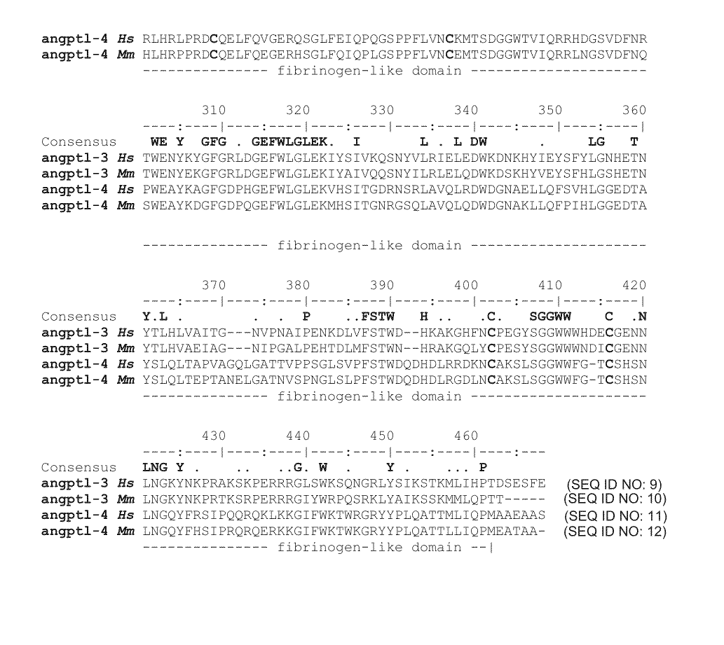

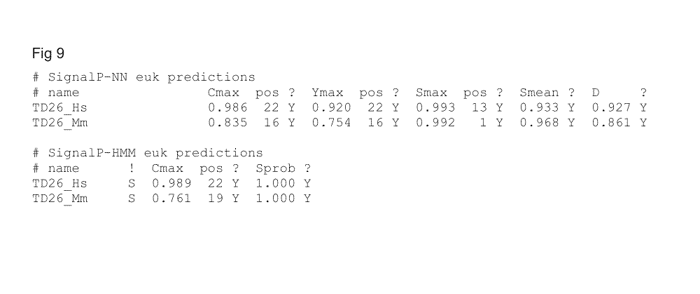

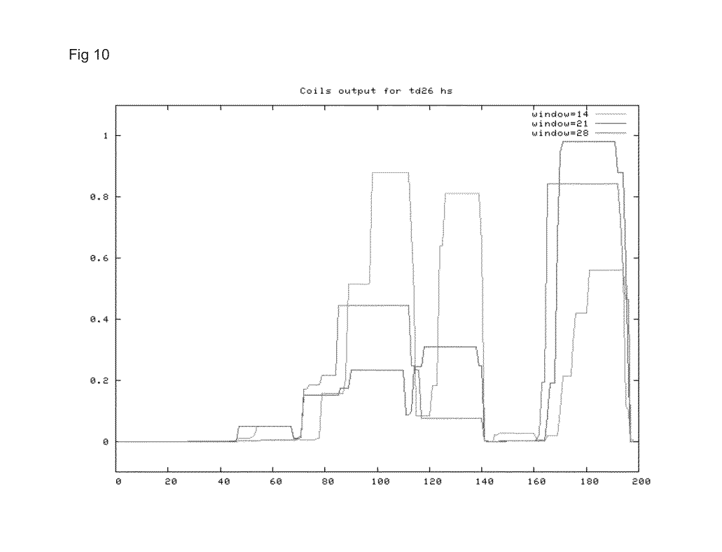

The database of Homo sapiens reference protein sequences (Database name: gp/9606.9558/hs_refp) at the “National Center for Biotechnology Information” (NCBI, http://www.ncbi.nlm.nih.gov) was searched for possible orthologs to TD26 using the blastp program (version 2.2.25+; Altschul at al., J. Mol. Biol. 215:403-410 (1990)) using NP_{—}061157.3 as probe and standard parameters. Only the E-value cutoff was raised to 1.0 to allow for the detection of more distantly related sequences. The search identified NP_{—}055310.1, angiopoietin-related protein 3 precursor [Homo sapiens] (Angptl3) as the only hit (besides the probe itself) with marginal significance (Expect=5e-06, Identities=40/182 (22%), FIG. 6). In parallel, Mus musculus reference protein sequence database (Database Name: gp/10090.9559/mm_refp) was searched using the same blast program and parameters and NP_{—}001074409.1 (mouse TD26 ortholog protein sequence) as probe. This search returned NP_{—}038941, angiopoietin-related protein 3 precursor [Mus musculus], as the only significant hit (Expect=3e-09, Identities=48/195 (25%), FIG. 7). Additionally there was a non-significant hit returned for NP_{—}065606.2, angiopoietin-related protein 4 [Mus musculus] (Angptl4) (Expect=0.36, identities=34/104 (33%). A multiple alignment of the protein sequences of TD26, Angptl3, and Angptl4 of Homo sapiens and Mus musculus was prepared using the “clustalw2” program (Larkin et al., Bioinformatics 23:2947-2948 (2007)) and hand optimized (FIG. 8). Overall the sequence conservation between the six sequences is low. There is a single region of higher conservation ranging from position 37 to 57 as shown in FIG. 8. This region overlaps with a region in Angptl3 and Angptl4 which is involved in the binding and inhibition of lipoprotein lipase (Lee et al., J Biol Chem. 284(20):13735-45 (May 15, 2009)). Three amino acid residues of Angptl4 within this region have been shown to be essential for the interaction with and inhibition of lipoprotein lipase (Yau et al., J Biol Chem. 284(18):11942-52 (May 1, 2009)). The corresponding amino acid residues in human and mouse TD26 are identical to those in human Angptl4 (position 48, 52, and 55 in FIG. 8).

The similarity of TD26 to a functionally important region of Angptl3 and Angptl4 on the background of low overall sequence similarity prompted an inquiry into whether there are other structural features in the proteins which might indicate a functional relation. Both Angptl3 and Angptl4 are secreted proteins, having an N-terminal signal peptide, an N-terminal coiled-coil domain (COD), a short linker and a C-terminal fibrinogen-like domain (FLD). The linker can be cleaved by proprotein convertases, releasing the CCD and the FLD as separate fragments into the circulation. Full length Angptls and their CCDs form di- or oligomers, while the FLD circulate as monomers (Miida & Hirayama, Curr Opin Lipidol. 2/(1):70-75 (February 2010)).

To determine whether TD26 is likely to have a signal peptide necessary for a secreted protein, the sequence was assessed using signal peptide prediction program SignalP (Emanuelsson et al., Nature Protocols 2:953-971 (2007)). Both algorithms employed by SignalP unequivocally predict both human and mouse TD26 to have signal peptides (FIG. 9). The similarity between TD26 and Angptl3, as shown in the blast output in FIG. 6, extends from amino acid residue 20 to the end of TD26, and from position 28 to 208 in Angptl3, covering the CCD of Angptl3. To examine if this region in TD26 may also have a coiled-coil structure, the sequence of TD26 was analyzed with the coiled-coil prediction program “Coils”, a web service of the Swiss Institute of Bioinformatics (Lupas, Meth. Enzymology 266:513-525 (1996)). The result for human TD26 is shown in FIG. 10. There are two possible regions of coiled-coil structure, ranging from position 79 to 140 and from position 165 to 194. While the prediction is not unambiguous for the first region, it is conclusive for the second region. Overall the predicted structure strongly resembles the CCD of Angptl3 and 4 (Miida & Hirayama, Curr Opin Lipidol. 2/(1):70-75 (February 2010)).

In summary, the sequence analysis shows that TD26 is a secreted protein showing all structural features identified so far in the CCD fragments of Angptl3 and Angptl4. Both proteins, and especially their CCD fragments, have been shown to act as inhibitors of Lipoprotein Lipase (LPL), affecting triglyceride-rich lipoprotein and HDL metabolism (Li, Curr Opin Lipidol. 17(2):152-156 (April 2006)). Since the amino acid residues of Angptl3 and Angptl4 involved in the inhibition of LPL activity are conserved in TD26, it is reasonable to assume that TD26 also plays a role in the regulation of LPL and triglyceride metabolism.

### Example 5

### In Vivo Effects of S661 on β-Cell Replication

During the course of work described herein, studies were performed to determine the in vivo effects of S661 on beta cell replication in normal as well as DIO (diet-induced obesity) mice treated for 4 days.

### Effects in Normal Mice

In a study, S661 was administered subcutaneously in vehicle to C57Bl/6 mice 3 times daily at the following doses 1 mg/kg; 0.5 mg/kg; 0.25 mg/kg or 0.125 mg/kg bodyweight for a period of 4 days. In parallel BrdU was administered once daily at a dose of 100 mg/kg for the same period. At the fifth day the pancreas was removed and fixed in PFA. Paraffin embedded sections were stained for insulin, DAPI and BrdU. The incorporation of BrdU in replicating beta cells was analyzed using an automated script on images generated on a Zeiss Axioimager Z2. Treatment with S661 in normal mice results in significantly increased beta cell replication (FIG. 12). For example, FIG. 12 shows that in comparison to vehicle alone, S661 treatment resulted in a 2-fold (3 times daily dose of S661 of 0.125 mg/kg) to a 5-fold (3 times daily dose of S661 of 1 mg/kg) increase in β-cell replication. These data suggest that low doses of S661 significantly increase beta cell replication in a dose-dependent manner.

In another study, S661 was administered subcutaneously in vehicle to C57Bl/6 mice as once daily injections of 1 mg/kg bodyweight for a period of 4 days. In parallel BrdU was administered once daily at a dose of 100 mg/kg for the same period. At the fifth day the pancreas was removed and the replication of beta cell was measured by flow cytometry. Treatment with S661 in normal mice results in significantly increased beta cell replication (FIG. 13). For example, FIG. 13 shows that S661 treatment resulted in about a 5 fold increase in the percentage of beta cell replication as compared to vehicle alone.

### Effects in DIO Mice (Diet-Induced Obesity)

C57Bl/6 mice were fed a high fat diet for 17 weeks. S661 was administered subcutaneously in vehicle to DIO mice at doses of 3 times 1 mg/kg or 0.125 mg/kg bodyweight for a period of 4 days. In parallel BrdU was administered once daily at a dose of 100 mg/kg for the same period. At the fifth day the pancreas was removed and fixed in PFA. Paraffin embedded sections were stained for insulin, DAPI and BrdU. The incorporation of BrdU in replicating cells was analyzed using an automated script on images generated on a Zeiss Axioimager Z2. Treatment with S661 in DIO mice results in an increased amount of beta cell replication and does not increase non-beta cell replication (FIG. 14A and FIG. 14B). Treatment with S661 in DIO mice also results in an increased islet area relative to total pancreas area (FIG. 14C).

### Example 6

### In Vivo Effects of TD26 on β-Cell Replication

During the course of work described herein, studies were performed in order to determine the in vivo effects of portions of TD26 polypeptide on beta cell replication. Deletion mutants of mouse TD26 polypeptide (FIG. 15) were each cloned into expression vectors containing a strong promoter driving the expression of a cDNA encoding the polypeptide and an N-terminus IgK signal peptide to facilitate secretion. It should be appreciated by those skilled in the art that the N-terminus IgK signal peptide is not present in the secreted deletion mutants. Plasmids were injected via tail vein into 8 week old male imprinting control region (ICR) mice. Ki67 was used as a marker for replication. The control was a plasmid encoding GFP, and beta cell replication rates were analyzed after 6 days. As shown in FIG. 15, portions of the TD26 protein were able to elicit beta cell replication.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### Sequences:

SEQ ID NO: 1—human TD26 amino acid sequence (includes predicted signal sequence); GENBANK™ Accession No. NP_{—}061157.3
SEQ ID NO: 2—mouse TD26 ortholog (EG624219) amino acid sequence (includes predicted signal sequence)
SEQ ID NO: 3—rat TD26 ortholog amino acid sequence (includes predicted signal sequence)
SEQ ID NO: 4—human/mouse/rat TD26 amino acid consensus sequence
SEQ ID NO: 5—human TD26 amino acid sequence (includes portion of predicted signal sequence)
SEQ ID NO: 6—human angiopoietin-related protein 3 precursor amino acid sequence
SEQ ID NO: 7—mouse TD26 ortholog (EG624219) amino acid sequence (includes portion of predicted signal sequence)
SEQ ID NO: 8—mouse angiopoietin-related protein 3 precursor amino acid sequence
SEQ ID NO: 9—human angiopoietin-related protein 3 precursor amino acid sequence (includes additional predicted signal sequence as compared with SEQ ID NO: 6)
SEQ ID NO: 10—mouse angiopoietin-related protein 3 precursor amino acid sequence (includes additional predicted signal sequence as compared with SEQ ID NO: 8)
SEQ ID NO: 11—human angiopoietin-related protein 4 precursor amino acid sequence
SEQ ID NO: 12—mouse angiopoietin-related protein 4 precursor amino acid sequence
SEQ ID NO: 13—intentionally skipped
SEQ ID NO: 14—human TD26 nucleic acid sequence (NCBI Ref NM_{—}018687.6)
SEQ ID NO: 15—mouse TD26 ortholog nucleic acid sequence (NCBI Ref NM_{—}001080940.1)
SEQ ID NO: 16—S661/S961 amino acid sequence
SEQ ID NO: 17—RB537 amino acid sequence
SEQ ID NO: 18—affinity-optimized portion of RB537/S661/S961 amino acid sequence
SEQ ID NO: 19—linker amino acid sequence
SEQ ID NO: 20—affinity-optimized portion of RB537 amino acid sequence
SEQ ID NO: 21—FLAG tag amino acid sequence
SEQ ID NO: 22—E-tag amino acid sequence
SEQ ID NO: 23—affinity-optimized portion of S661/S961 amino acid sequence
SEQ ID NO: 24—consensus sequence for affinity-optimized portion of RB537 and S661/S961 amino acid sequences

1. A method for increasing proliferation of pancreatic beta cells in a subject in need thereof, comprising administering to said subject an effective amount of an agent that increases level or activity of hepatocellular carcinoma-associated protein TD26 (TD26), thereby increasing proliferation of pancreatic beta cells.

2. A method according to claim 1, wherein increasing proliferation of pancreatic beta cells increases the level of endogenous insulin in the subject, thereby treating or preventing a disorder associated with a reduced level of endogenous insulin in the subject selected from the group consisting of metabolic syndrome, glucose intolerance, obesity and Type I or Type II diabetes.

3. A method according to claim 1, wherein increasing proliferation of pancreatic beta cells increases the level of endogenous insulin in the subject, thereby treating or preventing a disorder associated with resistance to endogenous insulin in the subject selected from the group consisting of metabolic syndrome, glucose intolerance, obesity and Type I or Type II diabetes.

4. A method for increasing proliferation of pancreatic beta cells in a subject or treating or preventing diabetes in a subject comprising administering to said subject an effective amount of an agent which is hepatocellular carcinoma-associated protein TD26 (TD26) or a functional portion thereof or a nucleic acid encoding TD26 or a functional portion thereof, thereby increasing the level of endogenous insulin in said subject.

5. A method according to claim 1, wherein said agent increases the level or activity of endogenous TD26 in said subject.

6. A method according to claim 1, wherein said agent increases expression or secretion of TD26.

7. (canceled)

8. A method according to claim 1, wherein said agent is TD26 protein or a functional portion thereof.

9. A method according to claim 8, wherein said functional portion does not comprise the complete amino acid sequence or the native signal peptide sequence of TD26.

10. A method according to claim 8, wherein said functional portion comprises a peptide that lacks one or more functional or intact domains of TD26 selected from the group consisting of a functional or intact LPL domain of TD26 and a functional or intact CCD domain of TD26.

11.-12. (canceled)

13. A method according to claim 8, wherein said functional portion comprises a peptide that lacks a functional or intact IVS of TD26.

14. A method according to claim 8, wherein said functional portion comprises a peptide selected from the group consisting of a peptide of amino acids 22 to 76 of SEQ ID NO: 1, a peptide of amino acids 48 to 76 of SEQ ID NO: 1, and a peptide of amino acids 77 to 135 of SEQ ID NO: 1.

15. A method according to claim 1, wherein said agent is a nucleic acid encoding TD26 protein or a functional portion of TD26.

16. A method according to claim 15, wherein said nucleic acid encodes a functional portion of the TD26 protein which does not comprise the complete amino acid sequence or native signal peptide of TD26.

17. A method according to claim 15, wherein said nucleic acid encodes a functional portion of the TD26 protein that lacks one or more functional or intact domains of TD26 selected from the group consisting of a functional or intact LPL domain of TD26 and a functional or intact CCD domain of TD26.

18.-19. (canceled)

20. A method according to claim 15, wherein said nucleic acid encodes a functional portion of the TD26 protein that lacks a functional or intact IVS of TD26.

21. A method according to claim 15, wherein said nucleic acid encoding a functional portion of TD26 comprises a nucleic acid encoding a peptide selected from the group consisting of a peptide of amino acids 22 to 76 of SEQ ID NO: 1, a peptide of amino acids 48 to 76 of SEQ ID NO:1, and a peptide of amino acids 77 to 135 of SEQ ID NO: 1.

22. A method according to claim 8, wherein said TD26 protein lacks a signal sequence.

23. A method according to claim 8, wherein said functional portion comprises a coiled-coil domain of TD26 protein.

24. A method according to claim 8, wherein said TD26 protein comprises all or a portion of SEQ ID NO: 1, SEQ ID NO: 2, SEQ ID NO: 3 or SEQ ID NO: 4, or an amino acid sequence at least 80% identical to SEQ ID NO: 1, amino acids 22-198 of SEQ ID NO: 1, SEQ ID NO: 2 or SEQ ID NO: 3.

25.-26. (canceled)

27. A method according to claim 15, wherein said nucleic acid comprises all or a portion of SEQ ID NO: 14 or SEQ ID NO: 15, or a nucleotide sequence at least 80% identical to SEQ ID NO: 14 or SEQ ID NO: 15.

28.-29. (canceled)

30. A method according to claim 1, wherein said agent is an insulin receptor antagonist selected from the group consisting of S661, a functional portion of S661, S961, a functional portion of S961, RB537, and a functional portion of RB537.

31.-33. (canceled)

34. A method according to claim 1, wherein said agent is a peptide selected from the group consisting of a peptide having an amino acid sequence at least 80% identical to SEQ ID NO: 16 and a peptide having an amino acid sequence at least 80% identical to SEQ ID NO: 17.

35.-37. (canceled)

38. A method according to claim 1 wherein increasing proliferation of beta cells causes beta cell mass to increase in said subject.

39. A method for increasing proliferation of pancreatic beta cells in a subject in need thereof, comprising administering to said subject an effective amount of an insulin receptor antagonist.

40.-42. (canceled)

43. A method of identifying a candidate agent that is capable of increasing proliferation of pancreatic beta cells, comprising assessing the ability of said candidate agent to antagonize the insulin receptor.

44. A method of identifying a candidate therapeutic agent for increasing proliferation of pancreatic beta cells comprising:
contacting a suitable cell with a test agent; and
determining the effect of said test agent on level or activity of TD26,
wherein a test agent which increases TD26 level or activity is a candidate therapeutic agent for increasing proliferation of pancreatic beta cells.

45.-50. (canceled)

51. An agent which is TD26 protein or a functional portion thereof or a nucleic acid encoding TD26 protein or a functional portion of TD26, wherein the agent increases beta cell proliferation.

52.-53. (canceled)

54. An agent according to claim 51, wherein said TD26 protein: (i) lacks a signal sequence or (ii) comprises a sequence selected from the group consisting of SEQ ID NO: 1, amino acids 22-198 of SEQ ID NO: 1, SEQ ID NO: 2, SEQ ID NO: 3, SEQ ID NO: 4, and an amino acid sequence at least 80% identical to SEQ ID NO: 1, amino acids 22-198 of SEQ ID NO: 1, SEQ ID NO: 2 or SEQ ID NO: 3, and wherein said function portion comprises a coiled-coil domain of TD26 protein.

55.-58. (canceled)

59. An agent according to claim 51, wherein said nucleic acid comprises all or a portion of SEQ ID NO: 14 or SEQ ID NO: 15, or a nucleotide sequence at least 80% identical to SEQ ID NO: 14 or SEQ ID NO: 15.

60.-70. (canceled)

71. A method of diagnosing a TD26-related disorder in a test individual comprising:
determining a TD26 level in a sample obtained from said test individual, wherein a TD26 level that is increased or decreased in said test individual compared to a TD26 level in a normal individual is indicative of a TD26-related disorder.

72.-74. (canceled)

75. A composition comprising an agent which increases beta cell proliferation selected from the group consisting of (i) a functional portion of S961 nucleic acid sequence or amino acid sequence which increases beta cell proliferation, (ii) a functional portion of S661 nucleic acid sequence or amino acid sequence which increases beta cell proliferation, (iii) a functional portion of RB537 nucleic acid sequence or amino acid sequence which increases beta cell proliferation, (iv) a functional portion of TD26 nucleic acid sequence or amino acid sequence which does not comprise the complete amino acid sequence of TD26 lacking its signal peptide, and which increases beta cell proliferation, and (v) a functional portion of TD26 peptide or a nucleic acid encoding the functional portion, wherein the functional portion is capable of increasing beta cell proliferation.

76.-87. (canceled)

88. A composition comprising a peptide that increases the proliferation of beta cells selected from the group consisting of a peptide of amino acids 22 to 76 of SEQ ID NO: 1, a peptide of amino acids 48 to 76 of SEQ ID NO: 1, a peptide of amino acids 77 to 135 of SEQ ID NO: 1, and combinations thereof, or a nucleic acid encoding any of the peptides.


SEQUENCE LISTING (25 sequences)

SEQ ID NO: 1 - protein, 198 aa, Homo sapiens
MPVPALCLLWALAMVTRPASAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRSAWLGPAYREFEVLKAHADKQSHILWALTGHVQRQRREMVAQQHRLRQIQERLHTAALPA

SEQ ID NO: 2 - protein, 198 aa, Mus musculus
MAVLALCLLWTLASAVRPAPVAPLGGPEPAQYEELTLLFHGALQLGQALNGVYRATEARLTEAGHSLGLYDRALEFLGTEVRQGQDATQELRTSLSEIQVEEDALHLRAEATARSLGEVARAQQALRDTVRRLQVQLRGAWLGQAHQEFETLKARADKQSHLLWALTGHVQRQQREMAEQQQWLRQIQQRLHTAALPA

SEQ ID NO: 3 - protein, 198 aa, Rattus norvegicus
MVVPILCLLWAIATAVRPAPVAPLGGPEPAQYEELTLLFHGALQLGQALNGVYKATEARLTEAGRNLGLFDQALEFLGREVNQGRDATRELRTSLSEIQAEEDTLHLRAEATARSLREVARAQHALRNSVRRLQVQLRGAWLGQAHQEFENLKDRADKQNHLLWALTGHVQRQQREMAEQQQWLRQIQQRLHMAALPA

SEQ ID NO: 4 - protein, 198 aa, Artificial
  Consensus sequence
  misc_feature (2)..(2): Xaa = Pro, Ala or Val
  misc_feature (14)..(14): Xaa = Met, Ser or Thr
  misc_feature (65)..(65): Xaa = Asn, His or Arg
  misc_feature (79)..(79): Xaa = Gln, Thr or Arg
  misc_feature (82)..(82): Xaa = Ser, Arg or Asn
  misc_feature (100)..(100): Xaa = Met, Val or Ala
  misc_feature (104)..(104): Xaa = Ile, Ala or Thr
  misc_feature (124)..(124): Xaa = Lys, Gln or His
  misc_feature (151)..(151): Xaa = Val, Thr or Asn
MXVPALCLLWALAXAVRPAPVAPLGGPEPAQYEELTLLFHGALQLGQALNGVYRATEARLTEAGXSLGLYDRALEFLGXEVXQGRDATQELRTSLSEIQXEEDXLHLRAEATARSLGEVARAQXALRDSVRRLQVQLRGAWLGQAHQEFEXLKARADKQSHLLWALTGHVQRQQREMAEQQQWLRQIQQRLHTAALPA

SEQ ID NO: 5 - protein, 236 aa, Homo sapiens
SAAPMGGPELAQHEELTLLFHGTLQLGQALNGVYRTTEGRLTKARNSLGLYGRTIELLGQEVSRGRDAAQELRASLLETQMEEDILQLQAEATAEVLGEVAQAQKVLRDSVQRLEVQLRQTSEIKEEEKELRRTTYKLQVKNEEVKNMSLELNSKLESLLEEKILLQQKVKYLEEQLTSAWLGPAYREFEVLKAHADKQSHILWALTGHVQRQRREMVAQQHRLRQIQERLHTAAL

SEQ ID NO: 6 - protein, 181 aa, Homo sapiens
SLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSI

SEQ ID NO: 7 - protein, 185 aa, Mus musculus
LASAVRPAPVAPLGGPEPAQYEELTLLFHGALQLGQALNGVYRATEARLTEAGHSLGLYDRALEFLGTEVRQGQDATQELRTSLSEIQVEEDALHLRAEATARSLGEVARAQQALRDTVRRLQVQLRGAWLGQAHQEFETLKARADKQSHLLWALTGHVQRQQREMAEQQQWLRQIQQRLHTAAL

SEQ ID NO: 8 - protein, 194 aa, Mus musculus
IASRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGI

SEQ ID NO: 9 - protein, 460 aa, Homo sapiens
MFTIKLLLFIVPLVISSRIDQDNSSFDSLSPEPKSRFAMLDDVKILANGLLQLGHGLKDFVHKTKGQINDIFQKLNIFDQSFYDLSLQTSEIKEEEKELRRTTYKLQVKNEEVKNMSLELNSKLESLLEEKILLQQKVKYLEEQLTNLIQNQPETPEHPEVTSLKTFVEKQDNSIKDLLQTVEDQYKQLNQQHSQIKEIENQLRRTSIQEPTEISLSSKPRAPRTTPFLQLNEIRNVKHDGIPAECTTIYNRGEHTSGMYAIRPSNSQVFHVYCDVISGSPWTLIQHRIDGSQNFNETWENYKYGFGRLDGEFWLGLEKIYSIVKQSNYVLRIELEDWKDNKHYIEYSFYLGNHETNYTLHLVAITGNVPNAIPENKDLVFSTWDHKAKGHFNCPEGYSGGWWWHDECGENNLNGKYNKPRAKSKPERRRGLSWKSQNGRLYSIKSTKMLIHPTDSESFE

SEQ ID NO: 10 - protein, 455 aa, Mus musculus
MHTIKLFLFVVPLVIASRVDPDLSSFDSAPSEPKSRFAMLDDVKILANGLLQLGHGLKDFVHKTKGQINDIFQKLNIFDQSFYDLSLRTNEIKEEEKELRRTTSTLQVKNEEVKNMSVELNSKLESLLEEKTALQHKVRALEEQLTNLILSPAGAQEHPEVTSLKSFVEQQDNSIRELLQSVEEQYKQLSQQHMQIKEIEKQLRKTGIQEPSENSLSSKSRAPRTTPPLQLNETENTEQDDLPADCSAVYNRGEHTSGVYTIKPRNSQGFNVYCDTQSGSPWTLIQHRKDGSQDFNETWENYEKGFGRLDGEFWLGLEKIYAIVQQSNYILRLELQDWKDSKHYVEYSFHLGSHETNYTLHVAEIAGNIPGALPEHTDLMFSTWNHRAKGQLYCPESYSGGWWWNDICGENNLNGKYNKPRTKSRPERRRGIYWRPQSRKLYAIKSSKMMLQPTT

SEQ ID NO: 11 - protein, 406 aa, Homo sapiens
MSGAPTAGAALMLCAATAVLLSAQGGPVQSKSPRFASWDEMNVLAHGLLQLGQGLREHAERTRSQLSALERRLSACGSACQGTEGSTDLPLAPESRVDPEVLHSLQTQLKAQNSRIQQLFHKVAQQQRHLEKQHLRIQHLQSQFGLLDHKHLDHEVAKPARRKRLPEMAQPVDPAHNVSRLHRLPRDCQELFQVGERQSGLFEIQPQGSPPFLVNCKMTSDGGWTVIQRRHDGSVDFNRPWEAYKAGFGDPHGEFWLGLEKVHSITGDRNSRLAVQLRDWDGNAELLQFSVHLGGEDTAYSLQLTAPVAGQLGATTVPPSGLSVPFSTWDQDHDLRRDKNCAKSLSGGWWFGTCSHSNLNGQYFRSIPQQRQKLKKGIFWKTWRGRYYPLQATTMLIQPMAAEAAS

SEQ ID NO: 12 - protein, 409 aa, Mus musculus
MRCAPTAGAALVLCAATAGLLSAQGRPAQPEPPRFASWDEMNLLAHGLLQLGHGLREHVERTRGQLGALERRMAACGNACQGPKGKDAPFKDSEDRVPEGQTPETLQSLQTQLKAQNSKIQQLFQKVAQQQRYLSKQNLRIQNLQSQIDLLAPTHLDNGVDKTSRGKRLPKMTQLIGLTPNATHLHRPPRDCQELFQEGERHSGLFQIQPLGSPPFLVNCEMTSDGGWTVIQRRLNGSVDFNQSWEAYKDGFGDPQGEFWLGLEKMHSITGNRGSQLAVQLQDWDGNAKLLQFPIHLGGEDTAYSLQLTEPTANELGATNVSPNGLSLPFSTWDQDHDLRGDLNCAKSLSGGWWFGTCSHSNLNGQYFHSIPRQRQERKKGIFWKTWKGRYYPLQATTLLIQPMEATAA

SEQ ID NO: 13 - skipped

SEQ ID NO: 14 - DNA, 597 bp, Homo sapiens
atgccagtgcctgctctgtgcctgctctgggccctggcaatggtgacccggcctgcctcagcggcccccatgggcggcccagaactggcacagcatgaggagctgaccctgctcttccatgggaccctgcagctgggccaggccctcaacggtgtgtacaggaccacggagggacggctgacaaaggccaggaacagcctgggtctctatggccgcacaatagaactcctggggcaggaggtcagccggggccgggatgcagcccaggaacttcgggcaagcctgttggagactcagatggaggaggatattctgcagctgcaggcagaggccacagctgaggtgctgggggaggtggcccaggcacagaaggtgctacgggacagcgtgcagcggctagaagtccagctgaggagcgcctggctgggccctgcctaccgagaatttgaggtcttaaaggctcacgctgacaagcagagccacatcctatgggccctcacaggccacgtgcagcggcagaggcgggagatggtggcacagcagcatcggctgcgacagatccaggagagactccacacagcggcgctcccagcctga

SEQ ID NO: 15 - DNA, 597 bp, Mus musculus
atggctgtgcttgctctctgcctcctgtggaccttagcatcagcagtgcgacccgctccagtggcccctctgggtggtccagagccagctcaatatgaagagctgaccctgctctttcacggggccctgcagctaggccaggccctcaatggcgtgtacagagccacagaggctcgcctgacagaagctgggcacagcctgggcctctatgacagagcactggaattcctggggacagaagtcaggcagggccaggatgccacacaggagcttcgcaccagcctgtcggagattcaggtggaagaggacgctttacaccttcgagctgaagccacagcccgatcactgggggaagtggcccgggcccagcaggctctgcgggacactgtacggagactacaagtgcagctgagaggcgcctggctcggtcaagcccaccaagaatttgagaccttaaaggctcgagctgataagcagagccacctcttatgggctctcactggccacgtgcagcgacagcagcgggagatggcagagcagcaacagtggctgcgacagatccagcagagactccacacagcagccctcccagcctga

SEQ ID NO: 16 - protein, 43 aa, Artificial
  S661/S961 peptide sequence
GSLDESFYDWFERQLGGGSGGSSLEEEWAQIQCEVWGRGCPSY

SEQ ID NO: 17 - protein, 70 aa, Artificial
  RB537 peptide sequence
MADYKDDDDKGSLDESFYDWFERQLGGGSGGSWLDQEWAWVQCEVYGRGCPSAAAGAPVPYPDPLEPRPG

SEQ ID NO: 18 - protein, 16 aa, Artificial
  Affinity-optimized peptide
GSLDESFYDWFERQLG

SEQ ID NO: 19 - protein, 6 aa, Artificial
  linker sequence
GGSGGS

SEQ ID NO: 20 - protein, 20 aa, Artificial
  Affinity-optimized peptide
WLDQEWAWVQCEVYGRGCPS

SEQ ID NO: 21 - protein, 8 aa, Artificial
  FLAG-epitope
DYKDDDDK

SEQ ID NO: 22 - protein, 13 aa, Artificial
  E-tag
GAPVPYPDPLEPR

SEQ ID NO: 23 - protein, 21 aa, Artificial
  Affinity-optimized peptide
SLEEEWAQIQCEVWGRGCPSY

SEQ ID NO: 24 - protein, 19 aa, Artificial
  consensus peptide
  misc_feature (2)..(2): Xaa = any amino acid
  misc_feature (3)..(3): Xaa = any amino acid
  misc_feature (7)..(8): Xaa = any amino acid
  misc_feature (13)..(13): Xaa = any amino acid
LXXEWAXXQCEVXGRGCPS

SEQ ID NO: 25 - protein, 9 aa, Artificial
  linker
GGSGGSGGS